(19) United States
(12) Patent Application Publication
(10) Pub. No.: US 2011/0097741 A1
(43) Pub. Date: Apr. 28, 2011

(54) PARTIAL T1R2 NUCLEIC ACID SEQUENCE, RECEPTOR PROTEIN AND ITS USE IN SCREENING ASSAYS

(76) Inventor: Jay Patrick Slack, Loveland, OH (US)

(21) Appl. No.: 12/297,848

(22) PCT Filed: Apr. 19, 2006
(86) PCT No.: PCT/CH2007/000186

§ 371 Date: Nov. 3, 2008

(51) Int. Cl. G01N 33/53; C07K 14/00; C12N 15/11; C12N 15/00; C12N 5/08; C12P 21/04

(52) U.S. Cl. 435/7.21; 530/350; 536/23.5; 435/320.1; 435/366; 435/69.1; 435/7.2; 435/7.32; 435/7.31

(57) ABSTRACT

A novel sweet receptor protein, corresponding nucleic acid sequence, expression vectors, transfected host cells, and screening methods for modulators including ligands of the sweet taste response employing the aforementioned are provided.

This is a Provisional Application For Patent pursuant to 35 U.S.C. §111(b).

The invention relates to assays based on a novel sweet receptor protein, heterologous expression systems containing nucleic acid constructs forming said sweet receptor protein, and the use of the sweet receptor protein in screening.

Assays with the novel taste receptor protein may be used to identify agents that can modulate the taste response in humans (sweet taste modulators), and thereby render certain foods more palatable or increase patient compliance in oral pharmaceutics and nutraceutics. Such sweet taste modulators include sweet tastants that elicit a taste response in humans.

The detection of sweet taste is known to be mediated by a receptor comprised of two subunits, T1R2 and T1R3, which are specifically expressed in taste receptor cells, and form a dimeric sweet taste receptor complex (T1R2/T1R3 heterodimer). Both subunits belong to the family of so-called “G-protein coupled receptors” or GPCRs, in particular class-C GPCRs.

Like most other GPCRs, the class-C receptors have a heptahelical transmembrane domain (TMD). However, unlike other types of GPCRs, the class-C GPCRs also have a large extracellular domain composed of two parts: a “venus flytrap module” (VFTM) that is involved in ligand binding; and a cysteine-rich domain (CRD), that contains nine highly conserved cysteines and that links the VFTM to the TMD. A variable length intracellular C-terminal tail completes the class-C receptor.

Activation of the sweet receptor response was thought to require both subunits of the dimeric sweet receptor complex, and to date, all sweeteners tested activate the T1R2/T1R3 heterodimer. Published tests conducted with the separate subunits of the human sweet receptor (T1R2 homomer or T1R3 homomer) have shown no activity, while the T1R2/T1R3 heterodimer responds to a broad spectrum of chemically diverse sweeteners, ranging from natural sugars (sucrose, fructose, glucose, maltose), sweet amino acids (D-tryptophan), and artificial sweeteners (acesulfame-K, aspartame, cyclamate, saccharin, sucralose), to sweet tasting proteins (monellin, thaumatin, brazzein) (compare for example Li et al. (2002), Proc Natl Acad Sci USA 99(7), 4692-6).

Studies of chimeric T1R receptors and site-directed mutagenesis indicate that the sweet compound cyclamate binds in the TMD of T1R3, thereby activating the heterodimeric T1R2/T1R3 receptor complex. Conversely, brazzein, a sweet protein, is reported to bind in the cysteine-rich domain of T1R3. thus activating the heterodimeric T1R2/T1R3 receptor complex.

T1R2 homomer binding assays are described in US 20050032158. Binding assays show binding only, as opposed to functional receptor activation, and are end-point-based and time consuming compared to faster functional assay that involve kinetic measurements. US 20050032158 further describes functional assays including cell-based assays for T1Rs, which are suitable for the known functional receptors T1R1/T1R3 and T1R2/T1R3.

To date, not only has there been no showing that the TMD of one of the TAS1 R monomers can not only bind sweet compounds but also activate G-proteins in the absence of the other obligate monomeric partner, but the presence of both subunits was believed to be essential for signal transduction.

Applicant has found that a novel receptor protein corresponding to a heavily truncated sequence of the T1R2 homomer of the T1R2/T1R3 heterodimer receptor complex forms, surprisingly, a functional sweet receptor that binds to a sweet ligand and is able to activate G-proteins.

The terms T1R2 “homomer” or “homomeric” polypeptide, protein, or receptor as used herein are meant to encompass the monomer, dimmer or oligomer of the T1R2 polypeptide or protein, as opposed to the heterodimeric T1R2, T1R3 receptor complex.

The novel receptor protein T1R2-TMD was found to have a different agonist spectrum than the full-length T1R2 homomer. The latter was surprisingly found to be able not only to bind to ligands but also to activate downstream signalling.

According to the methods provided herein, cells expressing both T1R2-TMD and a G-protein, but not T1R3 are contacted with test agents, optionally in combination with known or newly determined sweet tastants, to determine the properties of said agents as sweet taste modulators. The assays provided herein may therefore be used to identify a tested agent as sweet tastant or modulator of the sweet response (of the sweet tastant, T1R2, or downstream events).

The functional effects of the agent on the receptor and G-protein are determined by a suitable functional assay, for example, an assay that measures changes in parameters of the transduction pathways such as intracellular IP3 and Ca²⁺, or by other G-protein specific assays such as labeling with GTPγS, according to techniques known in the art.

Alternatively, binding assays may be used to determine ligand binding to T1R2-TMD.

In practising the various aspects and embodiments described herein in relation to cloning, elucidating ligand-receptor pairs, and finding modulators of the sweet response, recourse is made to conventional techniques in molecular biology, microbiology and recombinant technology. These include the various known methods suitable for G-protein coupled receptors (GPCRs) including T1R2-TMD. Accordingly, the skilled person is fully apprised of such techniques and as such they are hereafter treated only summarily in order to more fully describe the context of the present invention.

### SUMMARY

In a first aspect, provided is a T1R2-TMD sweet receptor able to bind to and be activated by perillartine comprising one or more of
a polypeptide substantially homologous to SEQ ID NO:2,
a polypeptide encoded by a nucleotide sequence substantially homologous to a nucleotide sequence set forth in SEQ ID NO:1 as determined by sequence identity,
a polypeptide encoded by a nucleotide sequence substantially homologous to a nucleotide sequence encoding the polypeptide set forth in SEQ ID NO:2 as determined by hybridisation,
a polypeptide encoded by a nucleotide sequence substantially homologous to a nucleotide sequence encoding the polypeptide set forth in SEQ ID NO:2 as determined by nucleotide sequence identity,
wherein the substantially homologous polypeptide has a sequence identity of at least 74%;
wherein the substantially homologous nucleotide as determined by sequence identity has a sequence identity of at least 65%; and
wherein the substantially homologous nucleotide as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;
with the proviso that the T1R2-TMD receptor does not comprise one or more of
   a polypeptide homologous to SEQ ID NO:10 or SEQ ID NO:12 ,
   a polypeptide encoded by a nucleotide sequence homologous to a nucleotide sequence set forth in SEQ ID NO:9 or SEQ ID NO:11 as determined by sequence identity,
   a polypeptide encoded by a nucleotide sequence homologous to a nucleotide sequence encoding the polypeptide set forth in SEQ ID NO:10 or SEQ ID NO:12 as determined by hybridisation,
   a polypeptide encoded by a nucleotide sequence homologous to a nucleotide sequence encoding the polypeptide set forth in SEQ ID NO:10 or SEQ ID NO:12 as determined by nucleotide sequence identity,
wherein the polypeptide homologous to SEQ ID NO:10 or SEQ ID NO:12 has a sequence identity of at least 60%;
wherein the nucleotide sequence homologous to a nucleotide sequence set forth in SEQ ID NO:9 or SEQ ID NO:11 as determined by sequence identity has a sequence identity of at least 50%; and
wherein the nucleotide sequence homologous to a nucleotide sequence set forth in SEQ ID NO:9 or SEQ ID NO:11 as determined by hybridisation hybridises under moderately stringent hybridization conditions at a temperature of 42° C. in a solution consisting of 50% formamide, 5×SSC, and 1% SDS, and washing at 42° C. in a solution consisting of 0.2×SSC and 0.1% SDS.

In another aspect, the invention is directed to a T1R2-TMD sweet receptor selected from the group consisting of

a polypeptide substantially homologous to SEQ ID NO:2,

a polypeptide encoded by a nucleotide sequence substantially homologous to a nucleotide sequence set forth in SEQ ID NO:1 as determined by sequence identity,

a polypeptide encoded by a nucleotide sequence substantially homologous to a nucleotide sequence encoding the polypeptide set forth in SEQ ID NO:2 as determined by hybridisation,

a polypeptide encoded by a nucleotide sequence substantially homologous to a nucleotide sequence encoding the polypeptide set forth in SEQ ID NO:2 as determined by nucleotide sequence identity,

wherein the substantially homologous polypeptide has a sequence identity of at least 74%;

wherein the substantially homologous nucleotide as determined by sequence identity has a sequence identity of at least 65%;

and wherein the substantially homologous nucleotide as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS.

In a further aspect, provided is a nucleic acid encoding a T1R2-TMD sweet receptor that is able to bind to and be activated by perillartine comprising one or more of

a nucleic acid substantially homologous to a nucleotide sequence set forth in SEQ ID NO:1 as determined by sequence identity,

a nucleic acid substantially homologous to a nucleotide sequence set forth in SEQ ID NO:1 as determined by hybridisation,

a nucleic acid substantially homologous to a nucleotide sequence encoding the T1R2-TMD sweet receptor,

wherein the substantially homologous nucleotide as determined by sequence identity has a sequence identity of at least 65%;

wherein the substantially homologous nucleotide as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;

with the proviso that the nucleic acid does not comprise one or more of

a nucleic acid homologous to a nucleotide sequence set forth in SEQ ID NO:9 or SEQ ID NO:11 as determined by sequence identity,

a nucleic acid homologous to a nucleotide sequence set forth in SEQ ID NO:9 or SEQ ID NO:11as determined by hybridisation,

a nucleic acid homologous to a nucleotide sequence encoding a polypeptide homologous to the polypeptide of SEQ ID NO:10 or SEQ ID NO:12,

wherein the homologous nucleotide as determined by sequence identity has a sequence identity of at least 50%, and

wherein the homologous nucleotide as determined by hybridisation hybridises under moderately stringent hybridization conditions at a temperature of 42° C. in a solution consisting of 50% formamide, 5×SSC, and 1% SDS, and washing at 42° C. in a solution consisting of 0.2×SSC and 0.1% SDS.

In another aspect, provided is a nucleic acid selected from the group consisting of

a nucleic acid substantially homologous to a nucleotide sequence set forth in SEQ ID NO:1 as determined by sequence identity,

a nucleic acid substantially homologous to a nucleotide sequence set forth in SEQ ID NO:1 as determined by hybridisation,

a nucleic acid substantially homologous to a nucleotide sequence encoding the T1R2-TMD sweet receptor,

wherein the substantially homologous nucleotide as determined by sequence identity has a sequence identity of at least 65%;

wherein the substantially homologous nucleotide as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS.

In another aspect, provided is an expression vector comprising the nucleic acid as defined herein-above.

In another aspect, provided is a host cell transfected with an expression vector as defined herein-above but that does not contain T1R3.

In certain illustrative embodiments, the host cell as defined herein-above stably expresses a T1R2-TMD sweet receptor as defined herein-above and a G-protein.

In other embodiments, the host cell as defined herein-above transiently expresses a T1R2-TMD sweet receptor as defined herein-above and a G-protein.

In another aspect, provided is a method of producing a T1R2-TMD sweet receptor as defined herein-above, the method comprising culturing host cells having contained therein an expression vector encoding for the T1R2-TMD sweet receptor, under conditions sufficient for expression, thereby forming the T1R2-TMD sweet receptor and optionally recovering it from the cells.

In another aspect, provided is a method to identify an agent that modulates sweet taste signaling in taste cells, the method comprises:

(i) contacting the cells that express a T1R2-TMD sweet receptor that responds to sweet taste stimuli with an agent, optionally in presence of another agent; and

(ii) determining whether at least one agent affects the functional activity of said T1R2-TMD sweet receptor in said cells by at least one functional response in said cells;

wherein said T1R2-TMD sweet receptor is selected from the group consisting of a polypeptide substantially homologous to SEQ ID NO:2, and a polypeptide encoded by a nucleotide substantially homologous to SEQ ID NO:1 as determined by sequence identity, and a polypeptide encoded by a nucleotide substantially homologous to SEQ ID NO:1 as determined by hybridisation;

wherein the substantially homologous polypeptide has a sequence identity of at least 74%;

wherein the substantially homologous nucleotide as determined by sequence identity has a sequence identity of at least 65%;

wherein the substantially homologous nucleotide as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;

and wherein the T1R2 sweet receptor expressing cells do not express a T1R3 receptor.

In certain illustrative embodiments, the method as defined herein-above utilizes cells that also express a G-protein.

In other embodiments, according to the method as defined herein-above, the G-protein is a chimeric G-protein based on Gaq-Gustducin.

In further embodiments, according to the method as defined herein-above, the G-protein is the chimeric G-protein Galpha16-gustducin 44.

In still further embodiments, according to the method as defined herein-above, (ii) is performed by measuring a change in or caused by intracellular messengers.

In yet further embodiments, according to the method as defined herein-above, the functional response is determined by measuring a change in an intracellular messenger selected from IP3 and calcium²⁺.

In other embodiments, according to the method as defined herein-above, the cells are selected from the group consisting of bacterial cells, eucaryotic cells, yeast cells, insect cells, mammalian cells, amphibian cells, and worm cells.

In certain embodiments, according to the method as defined herein-above, the cells are mammalian cells.

In further embodiments, according to the method as defined herein-above, the cells are mammalian cells selected from the group consisting of CHO, COS, HeLa and HEK-293 cells.

In still further embodiments, according to the method as defined herein-above, (i) further comprises contacting the T1R2 sweet receptor with a test agent in presence of a sweet tastant.

In yet further embodiments, according to the method as defined herein-above, the sweet tastant is selected from the group consisting of perillartine and methyl chavicol.

In another aspect, a kit is provided, the kit comprising:

(i) recombinant cells that express a T1R2-TMD sweet receptor, or a substantially similar homologue thereof, but that do not express a T1R3 receptor, and

(ii) an agonist of the T1R2-TMD sweet receptor,

for combined use to identify test agents as modulators of the T1R2-TMD.

In another aspect, provided is a method of using the kit defined herein-above. The method comprises:

(i) growing recombinant cells on a solid support;

(ii) adding test agents to a culture medium of defined plates or wells in the presence of the agonist in a suitable concentration, and

(iii) determining a change in a functional response of the cells by comparing the response in presence and absence of the test agent, and the test agent is thereby identified as a modulator of the T1R2 sweet receptor or a substantially similar homologue therof.

In another aspect, provided is a method to identify an agent that modulates T1R2-TMD, the method comprising:

(i) measuring a parameter that changes in response to ligand binding to T1R2-TMD

(ii) determining a change of the parameter in response to a test agent, optionally in presence of a ligand, in comparison to a negative control and thereby identifying a modulator or ligand.

In certain embodiments, according to the method as defined herein-above, the ligand is selected from the group consisting of perillartine and methyl chavicol.

In certain embodiments, according to the method as defined herein-above, (i) is performed by a method selected from the group consisting of fluorescence spectroscopy, NMR spectroscopy, measuring of one or more of absorbance, refractive index, hydrodynamic methods, chromatography, measuring solubility, biochemical, wherein the methods measure the properties of the T1R2-TMD polypeptide in a suitable environment selected form the group consisting of solution, bilayer membrane, attached to a solid phase, in a lipid monolayer, bound on a membrane, and in vesicles.

### DETAILED DESCRIPTION

### Cells Used in the Assays

Useful cells in screens or assays according to the invention are cells that contain no T1R3. Transfected or endogenous T1R3 can negatively interfere with methods that determine agonist responses of T1R2-TMD or the change of said responses dependent on another modulator. The absence of T1R3 provides a null background for the determination of T1R2-TMD activation, so that observed signals can be directly attributed to T1R2-TMD activity. This allows the identification of agents that specifically modulate T1R2-TMD, and excludes agents that activate T1R3 which could also include umami tastants, as T1R3 is part of both the sweet and the umami heterodimers.

Suitable eucaryotic cells include eucaryotic cells that do not contain T1R3, for example, without limitation, mammalian cells, yeast cells, or insect cells (including Sf9), amphibian cells (including melanophore cells), or worm cells including cells of Caenorhabditis (including Caenorhabditis elegans).

Suitable mammalian cells that do not contain T1R3, include, for example, without limitation, COS cells (including Cos-1 and Cos-7), CHO cells, HEK293 cells, HEK293T cells, HEK293 T-Rex™ cells, or other transfectable eucaryotic cell lines.

Suitable bacterial cells that do not contain T1R3 include without limitation E. coli.

Cells are transfected with a GPCR and a G-protein (which links the receptor to a phospholipase C signal transduction pathway) transiently or stably, as is well known in the art. An excellent heterologous expression system that employs the chimeric G-protein G alpha 16-gustducin 44 (also known as G.sub.alpha.16 gust(ducin)44, G.sub.alpha.16gust(ducin)44, Gα16gust(ducin)44, Ga16gust(ducin)44, G_{α16-gustducin 44}, or as used herein-below, “G16gust44”) which provides for enhanced coupling to taste GPCRs, is described in detail in WO 2004/055048. Alternatively, other chimeric G-proteins based on Gaq-Gustducin described in WO 2004/055048, or other G-Proteins, for example, G16 or G15, may also be used.

T1R2-TMD can be expressed in a cell with a G-protein that links the receptor to a signal transduction pathway, for example, the phospholipase C signal transduction pathway, or signal transduction pathways including, for example, the following: adenylate cyclase, guanylate cyclase, phospholipase C, IP3, GTPase/GTP binding, arachinoid acid, cAMP/cGMP, DAG, protein kinase c (PKC), MAP kinase tyrosine kinase, or ERK kinase.

Alternatively, any suitable reporter gene may be linked to a T1R2-TMD-activation responsive promoter and used to determine T1R2-TMD activity, as described in more detail herein-below.

### Vector Constructs Used in Cells Described Herein-Above

The vector constructs for expressing the GPCR and/or the G-protein in such cells may be produced in a manner known per se using Polymerase Chain Reactions. After verification of the sequence, cDNA fragments may be sub-cloned into a suitable vector, for example pcDNA 3.1 mammalian expression vector for mammalian cells, and transiently transfected in a mammalian host cell to enable the correct expression of the gene.

After a post-transfection period, for example 48 hours, cell lysates may be prepared, analysed by a Western-Blot analysis in order to confirm the correct expression of the protein. Once correct protein expression is confirmed, suitable cells, for example mammalian cells including HEK293T cells and HEK T-Rex™, may be transfected to generate cells stably expressing the protein according to techniques well known in the art.

Alternatively, a variety of non-mammalian expression vector/host systems can be used to contain and express sequences encoding the T1R2-TMD GPCR. These include, for example, microorganisms including bacteria transformed with recombinant bacteriophage, plasmid, or cosmid DNA expression vectors; yeast transformed with yeast expression vectors; insect cell systems infected with viral expression vectors (for example baculovirus), or with bacterial expression vectors (for example pBR322 plasmids).

Examples of specific vectors that may be used with the systems described herein-above are described in “G-protein coupled receptors (Signal Transduction Series)”; Editors: Tatsuya Haga and Gabriel Berstein, 1st ed., CRC Press—Boca Raton Fla.; September 1999.

In bacterial systems, a number of cloning and expression vectors may be selected depending upon the use intended for polynucleotide sequences encoding the GPCR. For example, routine cloning, subcloning, and propagation of polynucleotide sequences encoding a GPCR can be achieved using a multifunctional E. coli vector such as PBLUESCRIPT (Stratagene, La Jolla Calif.) or PSPORT1 plasmid (Life Technologies). Ligation of sequences encoding a GPCR into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. When large quantities of a GPCR are needed, for example, for the production of antibodies, vectors which direct high level expression of a GPCR may be used. For example, vectors containing the strong, inducible SP6 or T7 bacteriophage promoter may be used.

Yeast expression systems may be used for production of a GPCR. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation.

For the expression of heterologous proteins in insect cell lines is, for example, derivatives of the Lepidopteran baculovirus, Autographa californica multicapsid nucleo-virus (AcMNPV) can be used. In this system, foreign gene expression is directed by a very strong late viral promoter, either the polyhedrin or p10 promoters, and a wide array of vectors is available that optimises expression and recovery of recombinant proteins. These vectors enable expression of both membrane-bound and secreted proteins at high levels, and also many post-translational modifications known to occur in mammalian systems, including N- and O-linked glycosylation, phosphorylation, acylation, proteolysis and secreted vaccine components. A number of vectors are commercially available, for example the InsectSelect™ System from Invitrogen.

### Expression Systems

In order to express cDNAs encoding the desired proteins (GPCR and G-protein), one typically subclones the appropriate cDNA into an expression vector that contains a strong promoter to direct transcription, a transcription/translation terminator, and a ribosome-binding site for translational initiation. Suitable bacterial promoters are well known in the art, for example, E. coli, Bacillus sp., and Salmonella, and kits for such expression systems are commercially available. Similarly, eukaryotic expression systems for mammalian cells, yeast, and insect cells are commercially available. The eukaryotic expression vector may be, for example, an adenoviral vector, an adeno-associated vector, or a retroviral vector.

In addition to the promoter, the expression vector typically contains a transcription unit or expression cassette that contains all the additional elements required for the expression of the protein-encoding nucleic acid in host cells. A typical expression cassette thus contains a promoter operably linked to the nucleic acid sequence encoding the protein and signals required for efficient polyadenylation of the transcript, ribosome binding sites, and translation termination. The nucleic acid sequence encoding the protein may typically be linked to a membrane-targeting signal such as the N-terminal 45 amino acids of the rat Somatostatin-3 receptor sequence to promote efficient cell-surface expression of the recombinant protein, which is useful for cell-surface receptors. Additional elements may include, for example, enhancers.

An expression cassette should also contain a transcription termination region downstream of the structural gene to provide for efficient termination. The termination region may be obtained from the same gene as the promoter sequence or may be obtained from different genes.

For expression of the proteins, conventional vectors for expression in eucaryotic or procaryotic cells well known in the art may be used. Examples of vectors include bacterial expression vectors, for example, plasmids including pBR322-based plasmids, pSKF, and pET23D, and fusion expression systems, for example, GST and LacZ.

Expression vectors containing regulatory elements from eukaryotic viruses are typically used in eukaryotic expression vectors, for example SV40 vectors, cytomegalovirus vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, pcDNA3.1, pIRES and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, dihydrofolate reductase and the like.

The elements that are typically included in expression vectors may also include a replicon that functions in E. coli, a gene encoding drug resistance to permit selection of bacteria that harbor recombinant plasmids, and unique restriction sites in non-essential regions of the plasmid to allow insertion of eukaryotic sequences. The particular drug resistance gene chosen is not critical, any of the many drug resistance genes known in the art are suitable. The prokaryotic sequences are optionally chosen such that they do not interfere with the replication of the DNA in eukaryotic cells, if necessary.

In bacterial systems the GPCR cDNA fragment may be expressed alone or as a fusion protein wherein the GPCR of interest is fused to the E. coli periplasmic maltose-binding protein (MBP) wherein the MBP, including its signal peptide, is linked to the amino terminus of the GPCR. The wild-type GPCR cDNA or the MBP:GPCR fusion cDNA is subcloned into a suitable plasmid, for example pBR322, where in E. coli, GPCR expression is driven by the lac wild-type promoter. Methods of expression of GPCRs in E. coli are described, for example, in “G-protein coupled receptors (Signal Transduction Series)”; Editors: Tatsuya Haga and Gabriel Berstein, 1st ed., pp. 265-280 CRC Press—Boca Raton Fla.; September 1999.

Genetically engineered yeast systems and insect cell systems which lack endogenous GPCRs provide the advantage of a null background for T1R2-TMD activation screening.

Genetically engineered yeast systems substitute a human GPCR and Ga protein for the corresponding components of the endogenous yeast pheromone receptor pathway. Downstream signaling pathways are also modified so that the normal yeast response to the signal is converted to positive growth on selective media or to reporter gene expression (described by Broach, J. R. and J. Thorner (1996) Nature 384 (supp.):14-16).

Genetically engineered insect systems incorporate a human GPCR and Ga protein that enables receptor coupling the the phospholipase C signaling pathway (see for example Knight and Grigliatti, (2004) J Receptors and Signal Transduction 24: 241-256).

Amphibian cell systems, in particular melanophore cells, are described, for example, in WO 92/01810 that describes a GPCR expression system.

Overexpression of T1R2-TMD

T1R2-TMD may be overexpressed by placing it under the control of a strong constitutive promoter, for example the CMV early promoter. Alternatively, certain mutations of conserved GPCR amino acids or amino acid domains can be introduced to render the employed GPCR constitutively active.

Transfection of T1R2-TMD expression vector constructs into cells.

Standard transfection methods can be used to produce bacterial, mammalian, yeast or insect cell lines that express large quantities of the protein.

Any known method for introducing nucleotide sequences into host cells may be used. It is only necessary that the particular genetic engineering procedure used be capable of successfully introducing the relevant genes into the host cell capable of expressing the proteins of interest. These methods may involve introducing cloned genomic DNA, cDNA, synthetic DNA or other foreign genetic material into a host cell and include the use of calcium phosphate transfection, polybrene, protoplast fusion, electroporation, liposomes, microinjection, plasma vectors, viral vectors and the like.

For example, without limitation, the T-Rex™ expression system (Invitrogen Corp., Carlsbad, Calif.) may be used. The T-Rex™ System is a tetracycline-regulated mammalian expression system that uses regulatory elements from the E. coli Tn10-encoded tetracycline (Tet) resistance operon. Tetracycline regulation in the T-Rex™ System is based on the binding of tetracycline to the Tet repressor and derepression of the promoter controlling expression of the gene of interest.

Cell Culture

After transfection, the transfected cells may be cultured using standard culturing conditions well known in the art. It will be apparent to the skilled person that different cells require different culture conditions including approriate temperature and cell culture media.

T1R2-TMD Receptor Protein Recovery

If desired, the protein may be recovered from the cell culture using standard techniques. For example, the cells may be burst open either mechanically or by osmotic shock before being subject to precipitation and chromatography steps, the nature and sequence of which will depend on the particular recombinant material to be recovered. Alternatively, the recombinant protein may be recovered from the culture medium in which the recombinant cells had been cultured.

### Modulators that May be Identified by the Assays

Modulators (ligands, agonists, partial agonists, antagonists, inverse agonists, inhibitors, enhancers) of T1R2-TMD receptor activity can be identified as described herein below. There now follows a definition of the agents to be identified by said assays.

A modulator is an agent that effects an increase or decrease of one or more of the following: the cell surface expression of a receptor, the binding of a ligand to a receptor, the intracellular response initiated by an active form of the receptor (either in the presence or absence or an agonist). The modulator can itself be an agonist that binds to the receptor, activates it and thereby modulates an increase in the cellular response.

Modulators include various types of compounds, including small molecules, peptides, proteins, nucleic acids, antibodies or fragments thereof. These can be derived from various sources including synthetic or natural, extracts of natural material, for example from animal, mammalian, insect, plant, bacterial or fungal cell material or cultured cells, or conditioned medium of such cells.

A ligand is an agent that binds to the receptor; it may be an agonist, partial agonist, enhancer, antagonist, or inverse agonist.

An agonist (sweet tastant) is a ligand of the T1R2-TMD receptor that activates the receptor and increases an intracellular response when it binds to a receptor compared to the intracellular response in the absence of the agonist. Additionally or alternatively, an agonist may decrease internalization of a cell surface receptor such that the cell surface expression of a receptor is increased as compared to the number of cell surface receptors present on the surface of a cell in the absence of an agonist.

A partial agonist is an agonist that only partially activates the receptor in comparison to other agonists that maximally activate the receptor.

An antagonist is a ligand which binds to the receptor at the same (competitive antagonist) or at a different site (allosteric antagonist) as an agonist, but does not activate an intracellular response initiated by an active form of a receptor, thereby inhibiting the intracellular response induced by an agonist as compared to the intracellular response in the presence of an agonist and in the absence of an antagonist.

An inverse agonist, binding to a receptor, decreases the constitutive intracellular response mediated by a receptor as compared to the intracellular response in the absence of the inverse agonist.

An inhibitor decreases the binding of an agonist to the receptor as compared to the binding of the agonist in the absence of inhibitor, and/or decreases the intracellular response induced by an agonist.

An enhancer increases the binding of an agonist to the receptor as compared to the binding of the agonist in the absence of enhancer, and/or increases the intracellular response induced by an agonist.

The activity, or changes in activity, of a receptor binding a ligand and transmitting the signal through, for example, a G-protein (i.e. due to different interactions with modulators) can be determined by the assays described herein-below.

### Assays to Identify Modulators of the T1R2-TMD Receptor

Modulators can be identified using a wide variety of in vitro and in vivo assays to determine and compare functional effects/parameters, or alternatively by binding assays. The effects of the test agents upon the function of the receptors can be measured by examining a suitable functional parameters. Any physiological change that affects receptor activity can be used to identify modulators.

Such functional assays are well-known in the art, for example assays using intact cells or tissues isolated from animals based on measuring the concentration or activity or their change of a secondary messenger (including, for example, intracellular calcium (Ca²⁺), cAMP, cGMP, inositol phospate (IP₃), diacylglycerol/DAG, arachinoid acid, MAP kinase or tyrosine kinase), ion flux, phosphorylation levels, transcription levels, neurotransmitter levels, and assays based on GTP-binding, GTPase, adenylate cyclase, phospholipid-breakdown, diacylglycerol, inositol triphosphate, arachidonic acid release, PKC, kinase and transcriptional reporters. Some suitable assays are, for example, described in WO 01 18050.

Receptor activation typically initiates subsequent intracellular events, for example, increases in second messengers, for example, IP₃, which releases intracellular stores of calcium ions. Activation of some G-protein coupled receptors stimulates the formation of inositol triphosphate (IP3) through phospholipase C-mediated hydrolysis of phosphatidylinositol. IP₃ in turn stimulates the release of intracellular calcium ion stores. Thus, a change in cytoplasmic calcium ion levels, or a change in second messenger levels such as IP₃ can be used to determine G-protein coupled receptor activity.

All functional assays may be performed by samples containing cells expressing the receptor on their surfaces or on isolated cell membrane fractions. Useful cells are described herein-above. Instead of samples with separate cells or cell membranes, tissues from transgenic animals may be used.

To identify a modulator which is not an agonist itself (e.g. an antagonist, partial agonist, inverse agonist, inhibitor, or enhancer), samples with and without test agent are compared. For example, a control (with agonist but without modulator) is assigned a relative receptor activity value of 100. A decrease in activity relative to the control identifies an inhibitor, antagonist or inverse agonist, an increase identifies an enhancer. Usually, an increase or decrease in the measured activity of 10% or more in a sample with test agent compared to a sample without test agent or compared to a sample with test agent but based on cells that do not express T1R2-TMD (mock-transfected cells) can be considered significant.

Identification of Agonists or Partial Agonists:

To identify an agonist or partial agonist, a sample with test agent is compared to a positive control with an agonist (for example perillartine or methyl chavicol), or alternatively/additionally, samples with and without test agent are compared in their receptor activity. For example, an agonist or partial agonist will have a biological activity corresponding to at least 10% of the maximal biological activity of the positive control sweet tastant when the agonist or partial agonist is present at 100 mM or less, for example it may have a maximal biologial activity comparable to the agonist or higher. Maximal biological activity is defined as the maximal achievable receptor response to an agonist, for example perillartine or methyl chavicol, that can be achieved within a given receptor assay format and this response fails to increase further despite application of increasing concentrations of that same agonist.

Alternatively, an increase in the measured activity of, for example, 10% or more in a sample with test agent is compared to a sample without test agent or is compared to a sample with test agent but based on cells that do not express T1R2-TMD (mock-transfected cells).

To identify antagonists, receptor activity in the presence of a known agonist with and without a test agent is compared. Antagonists show a reduction of agonist-stimulated receptor activity, for example by at least 10%.

To identify inverse agonists, receptor activity in the presence of a known agonist with and without a test agent is compared in samples comprising animals/cells/membranes that overexpress the receptor as described herein-above. Inverse agonists show a reduction of constitutive activity of the receptor, for example by at least 10%.

Various examples of suitable detection methods that measure T1R2-TMD receptor activity in assays described herein-above follow.

Detection of Changes of Cytoplasmatic Ions or Membrane Voltage:

Cells are loaded with ion sensitive dyes to report receptor activity, as described in detail in “G-protein coupled receptors (Signal Transduction Series)”, CRC Press 1999: 1^{st} Edition; Eds Haga and Berstein. Changes in the concentration of ions in the cytoplasm or membrane voltage are measured using an ion sensitive or membrane voltage fluorescent indicator, respectively.

Calcium Flux:

Intracellular calcium release induced by the activation of GPCRs is detected using cell-permeant dyes that bind to calcium. The calcium-bound dyes generate a fluorescence signal that is proportional to the rise in intracellular calcium. The methods allows for rapid and quantitative measurement of receptor activity.

Cells used are transfected cells that co-express the T1R2-TMD GPCR and a G-protein which allows for coupling to the phospholipase C pathway as described herein-above. Negative controls include cells or their membranes not expressing T1R2-TMD (mock transfected), to exclude possible non-specific effects of the candidate compound.

The calcium flux detection protocol is described in detail in “G-protein coupled receptors (Signal Transduction Series)”; Editors: Tatsuya Haga and Gabriel Berstein, 1st ed., 424 pp. CRC Press—Boca Raton Fla.; September 1999, and an adapted version with is summarised below:

Day 0: 96-well plates are seeded with 8.5K cells per well and maintained at 37° C. overnight in nutritive growth media.

Day 1: Cells are transfected using 150 ng of GPCR DNA and 0.3 μl of Lipofectamine 2000 (Invitrogen) per well. Transfected cells are maintained at 37° C. overnight in nutritive growth media.

Day 2: Growth media is discarded and cells are incubated for 1 hour (at room temperature in the dark) with 75 μl of calcium assay solution consisting of 1.5 μM Fluo-4 AM (Molecular Probes) and 2.5 μM probenicid dissolved in a Hanks balanced salts solution (HBSS) that has been supplemented with 10 mM Hepes, 200 μM calcium chloride and 0.1% bovine serum albumin, pH 7.4 at 37° C.

125 μl of wash buffer consisting of 2.5 mM probenicid dissolved in a Hanks balanced salts solution (HBSS) that has been supplemented with 10 mM Hepes, 200 μM calcium chloride and 0.1% bovine serum albumin, pH 7.4 at 37° C., is added to each well and the plate is further incubated for 30 minutes at room temperature in the dark.

Buffer solutions are discarded and plate is washed 3 times with 100 μl wash buffer and cells are reconstituted in 200 μl of wash buffer and incubated for 15 minutes at 37° C. The plate is placed in a fluorescent microplate reader, for example, the Flexstation (Molecular Devices) or the FLIPR (Molecular Devices) and receptor activation is initiated following addition of 20 μl of a 10× concentrated ligand stock solution. Fluorescence is continuously monitored for 15 seconds prior to ligand addition and for 45-110 seconds after ligand addition. Receptor activation levels are defined as by the two following equations: % Activation=(Maximum fluorescence−baseline fluorescence/baseline fluorescence)*100 or Fluorescence Increase=Maximum Fluorescence−baseline fluorescence, where baseline fluorescence represents the average fluorescence levels prior to ligand addition.

Useful cells are mammalian cells as described herein-above, for example HEK293T cells and HEK293 T-Rex™ cells. Cells may be transfected with a GPCR and a G-Protein transiently or stably as is well known in the art. An excellent heterologous expression system is described in detail in WO 2004/055048.

A calcium flux assay can be performed, for example, as described in example 1 herein-below.

The identification of a modulator is performed as described above subject to the following modifications. The signals are compared to the baseline level of T1R2-TMD activity obtained from recombinant cells expressing T1R2-TMD in the presence of an agonist but in the absence of a test agent. An increase or decrease in T1R2-TMD activity, for example of at least 2 fold, at least 5 fold, at least 10 fold , at least a 100 fold, or more identifies a modulator.

Alternatively, the identification involves an increase or decrease fluorescence intensity of, for example, 10% or more, when compared to a sample without modulator, or when compared to a sample with modulator but in cells that do not express the T1R2-TMD polypeptide (mock-transfected cells).

Adenylate Cyclase Activity:

Assays for adenylate cyclase activity are performed, for example, as described in detail by Kenimer & Nirenberg, 1981, Mol. Pharmacol. 20: 585-591. Reaction mixtures are incubated usually at 37° C. for less than 10 minutes. Following incubation, reaction mixtures are deproteinized by the addition of 0.9 ml of cold 6% trichloroacetic acid. Tubes are centrifuged and each supernatant solution is added to a Dowex AG50W-X4 column. The cAMP fraction from the column is eluted with 4 ml of 0.1 mM imidazole-HCl (pH 7.5) into a counting vial in order to measure the levels of cAMP generated following receptor activation by the agonist. Control reactions should also be performed using protein homogenate from cells that do not express a T1R2-TMD polypeptide.

IP3/Ca²⁺ Signals

In cells expressing G-proteins, signals corresponding to inositol triphosphate (IP3)/Ca²⁺ and thereby receptor activity can be detected using fluorescence. Cells expressing a GPCR may exhibit increased cytoplasmic calcium levels as a result of contribution from both intracellular stores and via activation of ion channels, in which case it may be desirable, although not necessary, to conduct such assays in calcium-free buffer, optionally supplemented with a chelating agent such as EDTA, to distinguish fluorescence response resulting from calcium release from internal stores.

Phospholipase C/Intracellular Ca²⁺ Signals

T1R2-TMD is expressed in a cell with a G-protein that links the receptor to a phospholipase C signal transduction pathway. Changes in intracellular Ca²⁺ concentration are measured, for example using fluorescent Ca²⁺ indicator dyes and/or fluorometric imaging.

GTPase/GTP Binding:

For a GPCR including T1R2-TMD, a measure of receptor activity is the binding of GTP by cell membranes containing the GPCR. Measured is the G-protein coupling to membranes by detecting the binding of labelled GTP.

Membranes isolated from cells expressing the receptor are incubated in a buffer containing 35S-GTPγS and unlabelled GDP. Active GTPase releases the label as inorganic phosphate, which is detected by separation of free inorganic phosphate in a 5% suspension of activated charcoal in 20 mM H₃PO₄, followed by scintillation counting. The mixture is incubated and unbound labelled GTP is removed by filtration onto GF/B filters. Bound and labelled GTP is measured by liquid scintillation counting. Controls include assays using membranes isolated from cells not expressing T1R2-TMD (mock-transfected), in order to exclude possible non-specific effects of the test agent. The method is described in detail by Traynor and Nahorski, 1995, Mol. Pharmacol. 47: 848-854.

To identify modulators, as described herein-above, a change (increase or decrease) of 10% or more in GTP binding or GTPase activity is usually sufficient. However, to identify agonists, the assays described herein-above are performed subject to the following modifications. An agent is identified as an agonist usually if the activity is at least 50% of that of a known agonist (for example perillartine) when the compound is present at 100 mM or less, for example 10 to 500 μM, for example about 100 μM, or if it will induce a level the same as or higher than that induced by a known agonist.

Microphysiometer or Biosensor

Such assays can be performed as described in detail in Hafner. 2000, Biosens. Bioelectron. 15: 149-158.

Arachinoid Acid

The intracellular level of arachinoid acid is employed as an indicator of receptor activity. Such a method is described in detail by Gijon et al., 2000, J. Biol. Chem., 275:20146-20156.

cAMP/cGMP:

Intracellular or extracellular cAMP is measured using a cAMP radioimmunoassay (RIA) or cAMP binding protein, for example as described by Horton & Baxendale, 1995, Methods Mol. Biol. 41: 91-105. Alternatively, a number of kits for the measurement of cAMP are commercially available, for example the High Efficiency Fluorescence Polarization-based homogeneous assay by LJL Biosystems and NEN Life Science Products. Alternatively, the intracellular or extracellular levels of cGMP may measured using an immunoassay. For example, the method described in Felley-Bosco et al., Am. J. Resp. Cell and Mol. Biol., 11:159-164 (1994), may be used to determine the level of cGMP. Alternatively an assay kit for measuring cAMP and/or cGMP as described in U.S. Pat. No. 4,115,538 can be used.

Negative controls with mock-transfected cells or extracts thereof to exclude possible non-specific effects of test agents may be used.

DAG/IP3:

Second messengers Diacylglycerol (DAG) and/or inositol triphosphate (IP3), which are released by Phospholipid breakdown, that is caused by receptor activity, can be detected and used as an indicator of T1R2-TMD activity, for example as described in Phospholipid Signalling Protocols, edited by Ian M. Bird, Totowa, N.J., Humana Press, 1998. Alternatively, kits for the measurement of inositol triphosphates are available commercially from Perkin Elmer and CisBio International.

Negative controls with mock-transfected cells or extracts thereof to exclude possible non-specific effects of test agents may be used.

PKC Activity:

Growth factor receptor tyrosine kinases can signal via a pathway involving activation of Protein Kinase C (PKC), which is a family of phospholipid- and calcium-activated protein kinases.

Increases in gene products induced by PKC show PKC activation and thereby receptor activity. These gene products include, for example, proto-oncogene transcription factor-encoding genes (including c-fos, c-myc and c-jun), proteases, protease inhibitors (including collagenase type I and plasminogen activator inhibitor), and adhesion molecules (including intracellular adhesion molecule I (ICAM I)).

PKC activity may be directly measured as described by Kikkawa et al., 1982, J. Biol. Chem. 257: 13341, where the phosphorylation of a PKC substrate peptide, which is subsequently separated by binding to phosphocellulose paper, is measured. It can be used to measure activity of purified kinase, or in crude cellular extracts. Protein kinase C sample can be diluted in 20 mM HEPES/2 mM DTT immediately prior to the assay.

An alternative assay can be performed using the Protein Kinase C Assay Kit commercially available by PanVera.

The above-described PKC assays are performed on extracts from cells expressing T1R2-TMD.

Alternatively, activity can be measured through the use of reporter gene constructs driven by the control sequences of genes activated by PKC activation.

Negative controls with mock-transfected cells or extracts thereof to exclude possible non-specific effects of test agents may be used.

MAP Kinase Activity:

MAP kinase activity can be measured using commercially available kits, for example, the p38 MAP Kinase assay kit by New England Biolabs, or the FlashPlate™ MAP Kinase assays by Perkin-Elmer Life Sciences. p42/44 MAP kinases or ERK1/2 can be measured to show T1R2-TMD activity when cells with Gq and Gi coupled GPCRs are used, and an ERK1/2 assay kit is commercially available by TGR Biosciences, which measures the phosphorylation of endogenous ERK1/2 kinases following GPCR activation.

Alternatively, direct measurements of tyrosine kinase activity through known synthetic or natural tyrosine kinase substrates and labelled phosphate are well known; the activity of other types of kinases (for example, Serine/Threonine kinases) can be measured similarly.

All kinase assays can be performed with both purified kinases and crude extracts prepared from cells expressing a T1R2-TMD polypeptide.

The substrates of kinases that are used can be either full-length protein or synthetic peptides representing the substrate. Pinna & Ruzzene (1996, Biochem. Biophys. Acta 1314: 191-225) lists a number of phosphorylation substrate sites useful for detecting kinase activities. A number of kinase substrate peptides are commercially available. One that is particularly useful is the “Src-related peptide,” RRLIEDAEYAARG (commercially available from Sigma), which is a substrate for many receptor and nonreceptor tyrosine kinases. Some methods require the binding of peptide substrates to filters, then the peptide substrates should have a net positive charge to facilitate binding. Generally, peptide substrates should have at least 2 basic residues and a free-amino terminus. Reactions generally use a peptide concentration of 0.7-1.5 mM.

Negative controls with mock-transfected cells or extracts thereof to exclude possible non-specific effects of test agents may be used.

Transcriptional Reporters/T1R2-TMD-Responsive Promoter/Reporter Gene

To identify modulators with reporter gene assays, an at least 2-fold increase or 10% decrease in the signal is significant. An agonist stimulates for example at least 2-fold, 5-fold, 10-fold or more when comparing activity in presence and absence of the test agent.

The intracellular signal initiated by binding of an agonist to T1R2-TMD sets in motion a cascade of intracellular events, the ultimate consequence of which is a rapid and detectable change in the transcription or translation of one or more genes.

The activity of the receptor can therefore be determined by measuring the expression of a reporter gene driven by a promoter responsive to T1R2-TMD activation.

A “promoter” as used herein is one or more transcriptional control elements or sequences necessary for receptor-mediated regulation of gene expression, including one or more of basal promoter, enhancers and transcription-factor binding sites necessary for receptor-regulated expression. Promoters responsive to the intracellular signals resulting from agonist binding to T1R2-TMD are selected and operatively linked to a corresponding promoter-controlled reporter gene whose transcription, translation or ultimate activity is readily detectable and measurable.

Reporter genes may be selected, for example, from luciferase, CAT, GFP, β-lactamase, β-galactosidase, and the so-called “immediate early” genes, c-fos proto-oncogene, transcription factor CREB, vasoactive intestinal peptide (VIP) gene, the somatostatin gene, the proenkephalin gene, the phosphoenolpyruvate carboxy-kinase (PEPCK) gene, genes responsive to NF-κB, and AP-1-responsive genes (including the genes for Fos and Jun, Fos-related antigens (Fra) 1 and 2, IκBα, ornithine decarboxylase, and annexins I and II).

Promoters will be selected according to the selected reporter gene, as will be apparent to the skilled person.

Luciferase, CAT, GFP, β-lactamase, β-galactosidase and assays for the detection of their products are well known in the art. Examples of further reporter genes are described herein-below.

The “immediate early” genes are suitable and are rapidly induced (for example within minutes of contact between the receptor and the effector protein or ligand). Desirable properties in reporter genes include one or more of the following: rapid responsiveness to ligand binding, low or undetectable expression in quiescent cells; induction that is transient and independent of new protein synthesis: subsequent shut-off of transcription requires new protein synthesis; and mRNAs transcribed from these genes which have a short half-life of several minutes to a few hours. Similarly, the promoter may have one, several or all of these properties.

The c-fos proto-oncogene is an example of a gene that is responsive to a number of different stimuli and has an rapid induction. The c-fos regulatory elements include a TATA box that is required for transcription initiation; two upstream elements for basal transcription, and an enhancer, which includes an element with dyad symmetry and which is required for induction by TPA, serum, EGF, and PMA. The 20 by c-fos transcriptional enhancer element located between −317 and −298 bp upstream from the c-fos mRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is responsive to levels of intracellular cAMP. Therefore, the activation of a receptor that signals via modulation of cAMP levels can be determined by detecting either the binding of the transcription factor, or the expression of a reporter gene linked to a CREB-binding element (termed the CRE, or cAMP response element). The DNA sequence of the CRE is TGACGTCA. Reporter constructs responsive to CREB binding activity are described in U.S. Pat. No. 5,919,649.

Other suitable reporter genes and their promoters include the vasoactive intestinal peptide (VIP) gene and its promoter which is cAMP responsive; the somatostatin gene and its promoter which is cAMP responsive; the proenkephalin and its promoter which is responsive to cAMP, nicotinic agonists, and phorbol esters; and the phosphoenolpyruvate carboxy-kinase (PEPCK) gene and its promoter which is cAMP responsive.

Additional examples of reporter genes and their promoters that are responsive to changes in GPCR activity include the AP-1 transcription factor and NF-κB.

The AP-1 promoter is characterised by a consensus AP-1 binding site which is the palindrome TGA(C/G)TCA. The AP-1 site is also responsible for mediating induction by tumor promoters including the phorbol ester 12-O-tetradecanoylphorbol-β-acetate (TPA), and are therefore sometimes also referred to as a TRE, for TPA-response element. AP-1 activates numerous genes that are involved in the early response of cells to growth stimuli. Examples of AP-1-responsive genes include the genes for Fos and Jun (which proteins themselves make up AP-1 activity), Fos-related antigens (Fra) 1 and 2, IκBα, ornithine decarboxylase, and annexins I and II.

The NF-κB promoter/binding element has the consensus sequence GGGGACTTTCC. A large number of genes have been identified as NF-κB responsive, and their control elements can be linked to a reporter gene to monitor GPCR activity. Genes responsive to NF-κB include for example those encoding IL-1β, TNF-α, CCR5, P-selection, Fas ligand, GM-CSF and IκBα. Vectors encoding NF-κB-responsive reporters are known in the art or can be readily formed using ordinary skill in the art, for example, synthetic NF-κB elements and a minimal promoter, or using the NF-κB-responsive sequences of a gene known to be subject to NF-κB regulation. Further, NF-κB responsive reporter constructs are commercially available from, for example, CLONTECH.

A given promoter construct can easily be tested by exposing T1R2-TMD-expressing cells, transfected with the construct, to an agonist (for example perillartine). An increase of at least 2-fold in the expression of reporter gene in response to the agonist indicates that the reporter is suitable to measure T1R2-TMD activity.

Controls for transcription assays include both cells not expressing T1R2-TMD, but carrying the reporter construct, and cells with a promoterless reporter construct.

Agents that modulate T1R2-TMD activity as shown by reporter gene activation can be verified by using other promoters and/or other receptors to verify T1R2-TMD specificity of the signal and determine the spectrum of their activity, thereby excluding any non-specific signals, for example non-specific signals via the reporter gene pathway.

Inositol Phosphates (IP) Measurement:

Phosphatidyl inositol (PI) hydrolysis may be determined as described in U.S. Pat. No. 5,436,128, which involves labelling of cells with 3H-myoinositol for at least 48 hours or more. The labelled cells are contacted with a test agent for one hour, then these cells are lysed and extracted in chloroform-methanol-water. This is followed by separating the inositol phosphates by ion exchange chromatography and quantifying them by scintillation counting. For agonists, fold stimulation is determined by calculating the ratio of counts per minute (cpm) in the presence of tested agent, to cpm in the presence of buffer control. Likewise, for inhibitors, antagonists and inverse agonists, fold inhibition is determined by calculating the ratio of cpm in the presence of test agent, to cpm in the presence of buffer control (which may or may not contain an agonist).

### Binding Assays

Alternatively to the functional assays described herein-above that measure a change in parameters caused by a funtional response to ligand binding, ligand binding may be determined by binding assays that measure the binding of a ligand to the T1R2-TMD receptor.

Binding assays are well known in the art and can be tested in solution, in a bilayer membrane, optionally attached to a solid phase, in a lipid monolayer, or in vesicles. Binding of a modulator to a T1R2-TMD polypeptide can be determined, for example, by measuring changes in spectroscopic characteristics (for example fluorescence, absorbance, or refractive index), hydrodynamic methods (employing for example shape), chromatography, measuring solubility properties of a T1R2-TMD polypeptide. In one embodiment, binding assays are biochemical and use membrane extracts from cells/tissue expressing recombinant T1R2-TMD polypeptides.

A binding assay may, for example, be performed as described for T1Rs by Adler et al. in US20050032158, paragraphs [0169] to [0198], therein called “in vitro binding assays”, in distinction from functional assays which in US20050032158 are called “cell based binding assays”.

### T1R2-TMD Receptor Polypeptide and Nucleic Acid, and Substantially Homologous Polypeptides and Nucleic Acids

The T1R2-TMD receptor useful in methods according to the invention may be the receptor of SEQ ID NO:2, or alternatively a receptor (or nucleotide sequence to form the T1R2-TMD receptor) which is subtantially homologous and remains functional (i.e. binds to ligands and is activated by ligands). Such homologous receptors may be, for example, an allelic variant of SEQ ID NO:2, or the corresponding homologous sequence of a different species including rat (about 77.9% amino acid sequence identity and about 81.2% nucleic acid identity), mouse (about 76.2% amino acid sequence identity and about 80.9% nucleic acid identity), dog (about 74.4% amino acid sequence identity and about 82.6% nucleic acid identity), or any other species having sufficient amino acid sequence identity to the human receptor.

Further, substantially homologous T1R2-TMD nucleotide or polypeptide sequences may be formed by conservative mutations and/or point mutations and include any conservatively modified variant as detailed below.

With respect to nucleic acid sequences, conservatively modified variants means nucleic acids which encode identical or essentially identical amino acid sequences (conservatively substituted amino acids, i.e. lysine switched to arginine and further examples as explained herein-below).

Because of the degeneracy of the genetic code, a large number of nucleic acids different in sequence but functionally identical encode any given polypeptide/protein. Such nucleic acid variations are “silent variations,” which are one species of conservatively modified variations. Each nucleic acid sequence which encodes a polypeptide also describes every possible silent variation of the nucleic acid. Therefore, each codon in a nucleic acid (except AUG, which is ordinarily the only codon for methionine, and TGG, which is ordinarily the only codon for tryptophan) can be modified to yield a functionally identical nucleic acid sequence that will produce an identical polypeptide. Accordingly, each silent variation of a nucleic acid which encodes a polypeptide is implicit in each given nucleic acid sequence.

With respect to amino acid sequences, amino acid substitutions may be introduced using known protocols of recombinant gene technology including PCR, gene cloning, site-directed mutagenesis of cDNA, transfection of host cells, and in-vitro transcription which may be used to introduce such changes to the T1R2-TMD sequence. The variants can then be screened for taste-cell-specific GPCR functional activity. Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gin or glu; met/leu or tyr or ile; phe/met or leu or tyr; ser/thr; thr/ser; trp/tyr; tyr/trp or phe; val/ile or leu.

An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (1); 5) Isoleucine (I), Leucine (L), Methionine (M), Valine (V); and 6) Phenylalanine (F), Tyrosine (Y), Tryptophan (W).

Another alternative guideline is to allow for all charged amino acids as conservative substitutions for each other whether they are positive or negative.

In addition, individual substitutions, deletions or additions that alter, add or delete a single amino acid or a small percentage (for example up to 26%, or up to 20%, or up to 10%) of amino acids in an encoded sequence are also considered to be conservatively modified variations.

Substantially homologous nucleotide or polypeptide sequences have the degree of sequence identity or hybridize under certain stringent hybridization conditions as indicated below.

% Sequence Identity:

A substantially homologous nucleotide sequence has a % sequence identity of at least 65%, at least 70%, at least 75%, at least 85%, at least 90%, at least 95%, or at least 98%.

A substantially homologous polypeptide sequence has a % sequence identity of at least 74%, at least 75%, at least 80%, at least 85%, at least 90%, at least 95% or at least 98%.

Calculation of % Sequence Identity is Determined as Follows.

BLAST (Basic Local Alignment Search Tool) is the heuristic search algorithm employed by the programs blastn which is available at http://www.ncbi.nlm.nih.gov.

To determine % identity of a nucleotide query sequence against another nucleotide sequence, Blastn is used, using default parameters of BLAST version 2.2.1.3, including an EXPECT (statistical significance threshold for reporting matches against database sequences) of 10, and DUST filtering.

To determine % identity of a polypeptide query sequence against another polypeptide sequence, Blastp is used, using default parameters of BLAST version 2.2.1.3, including an EXPECT of 10, and DUST filtering.

Stringent Hybridization Conditions:

Nucleotide sequences are considered substantially homologous provided that they are capable of selectively hybridizing to the nucleotide sequences presented herein, or to their complement, under stringent hybridisation conditions detailed below.

Stringent conditions are temperature of 42° C. in a solution consisting of 50% formamide, 5×SSC, and 1% SDS and washing at 65° C. in a solution consisting of 0.2×SSC and 0.1% SDS (1×SSC=0.15 M NaCl, 0.015 M Na3 Citrate pH 7.0).

Background hybridization may occur because of other nucleotide sequences present, for example, in the cDNA or genomic DNA library being screened.

A positive signal that is is at least 2 times the signal strength of the background, optionally 10 times the signal strength of the background hybridization, is considered a specific interaction with (i.e. selective hybridization to) the target DNA. Optionally, a signal that is less than 10 fold as intense as the specific interaction observed with the target DNA is considered background. The intensity of interaction may be measured, for example, by radiolabelling the probe, e.g. with 32P.

### Kit to Identify a Modulator

A kit, for example a screening kit or high throughput screening kit, that comprises recombinant cells that express the T1R2-TMD homomer, or a substantially homologous sequence thereto, but that do not express T1R3; and that comprises an agonist of the T1R2-TMD homomer, for example perillartine or methyl chavicol.

Optionally, the cells further comprise a G-protein for example for calcium signalling. Suitable G-proteins are known and described herein-above; the skilled person is aware how to introduce them to the cells if necessary. A very useful chimeric G-protein is Galpha16-gustducin 44.

The agonist is provided in suitable concentrations, for example 1 nM to 10 mM, or 0.1 microM to 1 milliM, for example 0.1 microM to 100 microM.

Optional kit components may include a suitable medium for culturing the recombinant cells provided, and a solid support to grow the cells on, for example, a cell culture dish or microtiter plate, these optional components will be readily available to the skilled person.

The Kit May be Used as Follows:

(i) The recombinant cells are grown on the solid support.

(ii) Test agents at concentrations from about 1 nM to 100 mM or more are added to the culture medium of defined plates or wells in the presence of the agonist in a suitable concentration

(iii) A change in a functional response of the cells is determined by comparing the response in presence and absence of the test agent, and the test agent is thereby identified as a modulator.

For example, (iii) may be performed according to any one of the assays described-herein above, in combination with any one of the detection methods that report receptor activity described herein-above. This may require specifically chosen or adapted recombinant cells, which are also described herein-above.

A suitable assay is, for example, the calcium flux assay to determine activation of T1R2-TMD and its change in response to a test agent.

Confirmation of Identified Modulators

A modulator identified by a method described herein-above may easily be confirmed by simple sensory experiments using a panel of flavorists or test persons to taste the identified modulators. The compounds are tasted e.g. in water to confirm sweet taste or together with sweet tastants in comparison to a negative control without modulator to confirm a modulator that enhances the sweet taste.

Large Scale Screening Assays

Transcriptional reporter assays and most cell-based assays described herein-above are well suited for screening libraries for agents that modulate T1R2-TMD activity.

The assays may be designed to screen large chemical libraries by automating the assay steps and providing compounds from any convenient source to the assays, which are typically run in parallel (for example in microtiter formats on microtiter plates in robotic assays).

Assays may be run in high throughput screening methods that involve providing a combinatorial chemical or peptide library containing a large number of potential modulators. Such libraries are then screened in one or more assays described herein-above to identify those library agents (particular chemical species or subclasses) that display the activity described herein-above. The modulators thus identified can be directly used or may serve as leads to identify further modulators by making and testing derivatives.

Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.).

Libraries of Test Agents:

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

A rare chemical library is available from Aldrich (Milwaukee, Wis.).

Libraries of natural compounds in the form of bacterial, fungal, plant and animal extracts are commercially available for example from Pan Laboratories (Bothell, Wash.) or MycoSearch (N.C.), or are readily produceable by methods well known in the art. Additionally, natural and synthetically produced libraries and compounds are readily modified through conventional chemical, physical, and biochemical means.

Other libraries include protein/expression libraries, cDNA libraries from natural sources, including, for example, foods, plants, animals, bacteria, libraries expressing randomly or systematically mutated variants of one or more polypeptides, genomic libraries in viral vectors that are used to express the mRNA content of one cell or tissue.

In a high throughput assay, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 modulators. If 1536 well plates are used, then a single plate can easily assay from about 100 to about 1500 different compounds. It is possible to assay several different plates per day; assay screens for up to about 6,000-20,000 different compounds is possible.

### Types of Test Agents that May be Tested for Their T1R2-TMD Modulating Effect in the Assay Methods

The test agents may be any agent including small chemical compounds, chemical polymers, biological polymers, peptides, proteins, sugars, carbohydrates, nucleic acids and lipids. An agent can be a synthetic compound, a mixture of compounds, a natural product or natural sample, for example plant extract, culture supernatant, or tissue sample.

As examples of sweet tastant compounds, or compounds that modify sweet taste there may be mentioned Theasaponin E1, Acesulfame K, Alitame, Aspartame, CH 401, Dulcin, Erythritol, Guanidine Sweetener, Isomalt, Isomaltosylfructoside, Isoraffinose, NC 174, Neotame, Phenylacetylglycyl-L-Lysine, Saccharin, SC 45647, sodium Cyclamate, Sorbitol, Sucralose, Sucrononic Acid, Suosan, Superaspartame, Methyl alpha-L-arabinoside, Methyl beta-L-arabinoside, Methyl beta-D-Glucoside, Methyl a-D-mannoside, Methyl beta-L-xylopyranoside, Methyl alpha-D-xyloside, Methyl alpha-D-Glucoside 2,3-Di-threonine, Methyl alpha-D-Glucoside 2,3-Di-isoleucine, Protocatechuic Acid, Cynarin, Glycyphyllin, Rebaudioside C, Abrusoside A, Abrusoside B, Abrusoside C, Abrusoside D, Abrusoside E, Apioglycyrrhizin, Araboglycyrrhizin, Baiyunoside, Brazzein, Bryodulcoside, Carnosifloside V, Carnosifloside VI, D. cumminsii, Cyclocarioside A, Cyclocarioside I, Dulcoside A, Fluorene-4-alpha, 6-dicarboxylic acid, 4-beta, 10-alpha-dimethyl-1,2,3,4,5,10-hexahydor-Gaudichaudioside A, Glycyrrhizic Acid, Hernandulcin, Hernandulcin, 4beta-hydroxy-Hesperitin-7-Glucoside Dihydrochalcone, Huangqioside E, Huangqioside E, 3-Hydroxyphloridzin, Kaempferol, 2,3-Dihydro-6-Methoxy 3-O-Acetate, Mabinlin Maltosyl-Alpha-(1,6)-Neohesperidin Dihydrochalcone, Mogroside IIE, Mogroside III, Mogroside IIIE, Mogroside IV, Mogroside V, 11-Oxo Mogroside V, Monatin, Monellin, Monoammonium Glycyrrhizinate (Mag), Mukurozioside lib, Naringin Dihydrochalcone, Neoastilbin, Neohesperidin Dihydrochalcone (NHDHC), Neomogroside, Osladin, Pentadin, Periandrin I, Periandrin II, Periandrin III, Periandrin IV, Periandrin V, Phlomisoside I, Phlorizin, Phyllodulcin, Polypodoside A, Potassium magnesium calcium glycyrrhizin, Pterocaryosides A, Pterocaryosides B, Quercetin, 2,3-Dihydro-3-O-Acetate, Quercetin, 2,3-Dihydro-6-Methoxy-Quercetin, 2,3-Dihydro-6-Methoxy-3-O-Acetate, Rebaudioside A, Rebaudioside B, Rubusoside, Scandenoside R6, Siamenoside I, Sodium glycyrrhizinate, Steviolbioside, Stevioside, Stevioside, alpha-Glycosyl Suavioside A, Suavioside B, Suavioside G, Suavioside H, Suavioside I, Suavioside J, Thaumatin, Triammonium Glycyrrhizinate (TAG), Trilobatin Selligueain A, Haematoxylin, Maltitol, Mannitol, Methyl alpha-D-Glucoside 2,3-Di-aspartic acid, Benzoic Acid, 2-(4-Dimethylaminobenzoyl)-Benzoic Acid, 2-Hydroxy-4-aminomethyl-Benzoic Acid, 2-(3-Hydroxy-4-Methoxybenzoyl)-Methyl beta-D-fructoside, Methyl alpha-D-galactoside, Methyl beta-D-galactoside, Curculin, Strogin 1, Strogin 2, Strogin 4, Miraculin, Phenylacetic Acid, 3,4-Dimethoxy-Aminobenzoic Acid, 3-Anisic Acid, Benzyl alcoho, 3-Amino-4-n-propoxyl, 3,4 -Caffeic Acid, Cinnamic Acid, Dihydroxycinnamic Acid, 2,4-Ferulic Acid, Hydrolyzed Guar Gum, Hydroxyaminobenzoic Acid, 2,4-Nigerooligosaccharides, Sugarcane Bagasse Extract, Dihydroxybenzoic Acid, 2,3-Dihydroxybenzoic Acid, 2,4-Coumaric Acid, p-Dihydroxybenzoic Acid, 3,5-Hydroxybenzoic Acid, 3-Gurmarin, Gymnemasaponin III, Gymnemasaponin IV, Gymnemasaponin V, Gymnemic Acid I, Gymnemic Acid II, Gymnemic Acid III, Gymnemic Acid IV, Hodulcin, Jujubasaponin II, Jujubasaponin III, Propionic Acid, (−)-2-(4-Methoxyphenoxy)-Ziziphin, Ethyl Maltol, Maltol, Butanoic Acid, 2-Oxo-3-Methyl-Alanine, N-(1-Methyl-4-oxo-2-imidazolin-2-yl) Creatinine, Abrusoside E, mono-methyl ester, Lactitol, Periandrinic acd I, monoglucuronide, Periandrinic acid II, monoglycuronide, Xylitol, Tagatose, d-Benzoyloxyacetic acid, 4-Methoxy Hoduloside I, 4-Nitrophenyl a-D-galactoside, 4-Nitrophenyl alpha-D-glucoside, 4-Nitrophenyl beta-D-glucoside, 4-Nitrophenyl alpha-D-mannopyranoside, Urea, (N-(4-cyanophenyl)-N′-((sodiosulfo)methyl)-Chloramphenicol, Chlorogenic Acid, Methyl alpha-D-Glucoside, Methyl alpha-D-Glucoside 2,3-Di-alanine, Methyl alpha-D-Glucoside 2,3-Di-glycine, Methyl alpha-D-Glucoside 2,3-Di-proline, Methyl alpha-D-Glucoside 2,3-Di-valine, Aniline, 2-Butoxy-5-Nitro-Aniline, 2-Ethoxy-5-Nitro-Aniline, 2-Methoxy-5-Nitro-Aniline, 3-Nitro-(+)-Baiyunol-beta-D-gluccoside-alpha-D-glucoside, Aniline, 1,3-Hydroxy-4-methoxybenzylAniline, 2-Propxy-5-Nitro-(P4000)Benzo-1,4-dioxane 2-(3-Hydroxy-4-Methoxyphenyl)-Benzoe-1,3-dioxan-4-one 2-(3-Hydroxy-4-methoxyphenyl)-Benzoic Acid, 2-Benzoyl-4-Methoxy-Benzoic Acid, 2-(4-Methoxybenzoyl)-Benzo-1,3(4H)-xathiane, 2-(3-Hydroxy-4-Methoxyphenyl)-Benzo-1,4-xathiane 3-(3-Hydroxy-4-methoxyphenyl)-Butanoic acid, 4-[3,5-dihydroxy-4-[3-(3-hydroxy-4-methoxyphenyl)-1-oxopropyl]phenoxy]-2-hydroxy-monosodium salt, Butanoic acid, 4-[3,5-dihydroxy-4-[3-(3-hydroxy-4-methoxyphenyl)-1-oxopropyl]phenoxy]-3-oxo-monosodium salt, Cyclohexadiene-1,4 1-Carboxaldehyde-4-(Methoxymethyl)-, (E)oxime Ethylbenzene, beta-(1,3-Hydroxy-4-methoxybenzyl)-Hespertin Dihydrochalcone, 3′-Carboxy-Hespertin Dihydrochalcone, 3′-Formyl-lsocoumarin, 3,4-Dihydro-3-(3-Hydroxy-4-methoxy)-Perillartine, 8,9-epoxy-Phenyl 3-Hydroxy-4-methoxybenzyl Ether, Phosphonic acid, [3-[3,5-dihydroxy-4-[3-(3-hydroxy-4-methoxyphenyl)-1-oxopropyl]phenoxy]propyl] monopotassium salt, Stevioside analogue, Sulfamic acid, [2-[3,5-dihydroxy-4-[3-(3-hydroxy-4-methoxyphenyl)-1-oxopropyl]phenoxy]ethyl]-monopotassium salt, Urea, and N-(4-cyanophenyl)-N′-(2-carboxyethyl)-L-Theanine.

Identified sweet tastants may include, for example, artificial sweeteners that are able to elicit a sweet taste sensation. These are of particular interest as they can be used to replace sugar compounds, for example, to reduce calories or to provide consumables that are more healthy for the teeth. Consumables include food products, beverages, oral care products, and compositions for admixture to such products, in particular flavour compositions. Flavour compositions may be added to processed foods or beverages during their processing, or they may actually be consumables in their own right, e.g. condiments such as sauces and the like. Sweet tastants are particularly interesting in confectionary and other sweet consumables including desserts, but also in savoury and sweet-sour consumables. Examples of consumables include confectionary products, cakes, cereal products, baker's products, bread products, gums, chewing gums, sauces (condiments), soups, processed foods, cooked fruits and vegetable products, meat and meat products, egg products, milk and dairy products, cheese products, butter and butter substitute products, milk substitute products, soy products, edible oils and fat products, medicaments, beverages, alcoholic drinks, beers, soft drinks, food extracts, plant extracts, meat extracts, condiments, sweeteners, nutraceuticals, pharmaceutical and non-pharmaceutical gums, tablets, lozenges, drops, emulsions, elixirs, syrups and other preparations for making beverages, instant beverages and effervescent tablets.

T1R2-TMD Sequences

The sequences are shown in the sequence listing herein-below. SEQ ID NO:1 corresponds to the nucleotide/nucleic acid sequence encoding the T1R2-TMD receptor, SEQ ID NO: 2 corresponds to the polypeptide/amino acid sequence of the T1R2-TMD receptor protein. In the transfected construct, the SST TAG (SEQ ID NO:3) is followed by the nucleic acid coding for the novel T1R2-TMD protein (SEQ ID NO:1), which is followed by the HSV TAG nucleic acid (SEQ ID NO:5).

The resulting protein will accordingly comprise the following amino acids in the order indicated: amino acids of SEQ ID NO:4, SEQ ID NO:2, and SEQ ID NO:6.

SEQ ID NOS: 1+2: T1R2-TMD nucleic acid and protein

SEQ ID NOS: 3+4: SST TAG nucleic acid and protein

SEQ ID NOS: 5+6: HSV TAG nucleic acid and protein

SEQ ID NOS: 7+8 Forward and reverse primer for T1R2-TMD vector contruct

SEQ ID NOS: 9+10 T1R2 full length (nucleic acid and protein)

SEQ ID NOS: 11+12 T1R3 full length (nucleic acid and protein)

There now follows a series of examples that serve to illustrate the above-described methods. The following examples are merely illustrative and should not be construed as limiting the methods or kit in any manner.

### EXAMPLES

All examples use the human receptors.

### Example 1

Fluo-4 Calcium Assay

Fluo-4 is a fluorescent indicator for intracellular calcium and allows to determine changes in the calcium concentration, in particular an increase in response to receptor activation occurring after ligand addition (for example perillartine or methyl chavicol).

HEK293 cells stably expressing G alpha 16 gustducin 44 and transfected with T1R2-TMD as described in example 3 were used as host cells.

Black, clear-bottom 96-well plates were used for all assays. They were seeded the day before with 8500 transfected cells per well and maintained at 37° C. overnight in a growth medium appropriate for the cells used. For HEK293 cells, Dulbecco's Modified Eagle medium containing high glucose, L-glutamine, pyroxidine hydrochloride, and supplemented with 10% fetal bovine serum was used for growth and maintenance of the HEK293 cells.

At the time of the assay, the growth medium was discarded and cells were incubated for 1 hour (at 37° C. in the dark) with 50 μl of a calcium assay solution consisting of 1.5 μM Fluo-4 AM (Molecular Probes™, Invitrogen, US) and 2.5 μM probenicid (Sigma-Aldrich) dissolved in a C1 buffer solution. C1 buffer solution contains 130 mM NaCl, 5 mM KCl, 10 mM Hepes, 2 mM CaCl2 and 10 mM glucose (pH 7.4).

After the initial 1 hour loading period, the plates were washed 5 times with 100 μl per well of C1 buffer using an automated plate washer (BioTek) and after washing, the plate was further incubated for 30 minutes at room temperature in the dark to allow for complete de-esterification of the Fluo-4-AM. The buffer solutions were discarded, the plate was washed 5 times with 100 μl C1 wash buffer and finally the cells were reconstituted in 180 μl of C1 wash buffer.

For assay reading, the plate was placed in a FLIPR (fluorescence imaging plate reader (FLIPR-Tetra, Molecular Devices)), and receptor activation was initiated following addition of 20 μl of a 10× concentrated ligand stock solution.

Fluorescence was continuously monitored for 15 seconds prior to ligand addition and for 105 seconds after ligand addition (45-105 sec may be sufficient).

Receptor activation is given in relative flourescence units (RFU) and is defined by the following equation:

Fluorescence Increase=Maximum Fluorescence−baseline fluorescence,

wherein the baseline fluorescence represents the mean fluorescence calculated for the first 10 to 15 seconds prior to ligand addition.

As a negative control, mock transfected cells were exposed to the same concentration of ligand and the concentration of calcium traces not corresponding to a signal was determined.

Cells with an activated receptor were identified by the signal (RFU) being significantly above the negative control.

### Example 2

Preparation of a T1R2-TMD Vector Construct

PCR using Pfu polymerase (Invitrogen) was used to generate the T1R2-TMD construct using the specific primers listed below:

T1R2-TMD Forward Primer:
5′-TAT AGA ATT CGC ACC CAC CAT CGC TGT GGC C-3′
T1R2-TMD Reverse Primer:
5′-ATA TGC GGC CGC AGT CCC TCC TCA TGG T-3′

The template for the PCR amplification was a full length cDNA for human T1R2 isolated from a cDNA library generated from human fungiform papillae taste tissue. Reaction parameters were: 94° C. for 5 min followed by 35 cycles of 94° C. for 45 seconds, 54° C. for 15 seconds and 68° C. for 1 minute, followed by a final extension cycle of 68° C. for 10 minutes.

The resulting nucleic acid fragment (compare Seq ID NO:1) was separated by gel electrophoresis, purified and subcloned into the pCR-Topo-II vector (Invitrogen) and the resulting clones were verified by DNA sequencing to ensure absence of mutations arising from the PCR amplification. After sequencing, the T1R2-TMD insert was subcloned into an expression cassette based on pcDNA4-TO (Invitrogen). The cloning cassette already contains the first 45 amino acids of the rat somatostatin type 3 receptor at the N-terminus to facilitate cell surface membrane targeting of the transgene (described by Bufe et al. 2002, Nat Genet, 32(3), 397-401). The C-terminus of this vector encodes the herpes simplex virus (HSV) glycoprotein D epitope, which can be used for immuncytochemistry studies using a specific antibody that binds to this epitope. The resulting vector construct allows for expression of the T1R2-TMD protein of joined amino acid sequences of Seq ID 2 (T1R2-TMD) preceded by Seq ID NO:4 (45 aminoacids of rat somatostatin) and followed by 6 (HSV epiotope) (in amino terminus to C terminus direction).

### Example 3

Transfection of T1R2-TMD into Cells, Cells Stably Expressing T1R2-TMD and G16qust44

Human cell lines that stably express human T1R2-TMD were generated by transfecting a linearised pCDNA 4-T0 vector (Invitrogen) containing the hT1R2-TMD (formed as described in 2) into a G16gust44 expressing cell line, which was formed as described in WO 2004/055048. This cell line shows enhanced coupling to taste receptors, is tetracycline inducible, stably expresses the G16gust44 promiscuous G-protein, and is based on the HEK-293-T-Rex cell line (commercially available from Invirtogen, USA).

Transfection is Performed as Follows:

On day 0, the HEK293T/G16gust44 cells are plated in 6-well black, clear-bottom plates at a density of 900,000 cells per well and grown overnight in selective growth media.

On day 1, the media is changed to an antibiotic-free and serum-free growth medium and the cells are transfected using 4 ug of linearized T1R2 TMD vector construct DNA and 0.3 l of Lipofectamine 2000 (Invitrogen. The lipofectamine/DNA mixture is incubated on the cells for 3-4 hours and then replaced with an antibiotic-free, serum-containing growth medium. After 24 hours the cells are re-plated in selective medium containing DMEM supplemented with 10% FBS, 0.005 mg/ml blasticidin, 0.36 mg/ml G418, and 0.1 mg/ml zeocin (Invivogen) at 37.degrees C. After 2-4 weeks zeocin-resistant colonies were selected, expanded, and tested by calcium imaging as described in example 1 for responses to 50 M perillartine.

Resistant colonies were expanded, and identified as containing T1R2-TMD by their response to 50 μM perillartine, which was determined via automated fluorimetric imaging on the FLIPR-Tetra instrumentation (Molecular Devices) using the methods described in example 1 following induction of T1R2-TMD expression with 10 mg/ml tetracycline.

All potential clones were also evaluated for a functional response to 50 μM perillartine in the absence of tetracycline induction to identify any clones that are basally expressing low-level but functionally sufficient levels of the T1R2-TMD receptor (The tetracycline-regulated systems such as the T-Rex HEK-293 (Invitrogen) are known to have a low-level basal expression of transgenes due to the inherent leakiness of the system.)

As a result of said evalution, it was determined that when these cell lines are exposed to perillartine, many of cell clones respond to this stimulus with a significant increase in fluorescence which is greater than 10-times of the signal compared to the signal of the negative control (cells that express solely G16gust44 promiscuous G-protein but not T1R2-TMD).

Signals are significantly lower in cells treated with tetracycline to induce overexpression of the T1R2-TMD.

The lack of response in the tetracycline-induced T1R2-TMD cells may be due to cellular toxicity arising from tetracycline-induced overexpression of the T1R2-TMD.

| | AVERAGE | STD |
|---|---|---|
| | [RFU] | [RFU] |
| T1R2-TMD/G16gust44 | 973.06 | 69.48 |
| G16gust44 ONLY | 73.78 | 55.43 |
| (neg. control) | | |

### Example 4

Transfection of Cells Stably Expressing the T1R2/T1R3 Sweet Receptor Heterodimer and G16gust 44

T1R3 was constitutively overexpressed in the presence of a tetracycline-regulated T1R2 to avoid possible cytotoxic effects of constitutive overexpression of both proteins. Placing one subunit of the heterodimer (T1R2) in a tetracycline-regulated vector allows to regulate its expression level so that viability and functionality of the stable clonal lines can be optimised accordingly.

Human cell lines that stably express the human T1R2/T1R3 sweet heterodimer were generated by first transfecting a linearized pIRES-Puro vector (Clontech) containing the human T1R3 into a G16gust44 expressing cell line, which was formed as described in WO 2004/055048. This cell line shows enhanced coupling to taste receptors, is tetracycline inducible, stably expresses the G16gust44 promiscuous G-protein, and is based on the HEK-293-T-Rex cell line (commercially available from Invirtogen, USA). After generation of a heterogenous population of cells stably expressing T1R3, a linearized pcDNA4-TO vector (Invitrogen) containing human T1R2 cDNA was transfected.

After 24 hours the cells were re-plated at 10× dilutions up to 1:150,000 in selective medium containing Gluatamax DMEM (Invitrogen) supplemented with 10% FBS, 0.005 mg/ml blasticidin. 0.36 mg/ml G418, and 0.4 μg/ml puromycin at 37.degrees C. After 2 weeks a puromycin-resistant heterogeneous population of T1R3-expressing cells were then transfected with 4 ug of the linearized T1R2 vector construct DNA and 0.3 l of Lipofectamine 2000 (Invitrogen). The lipofectamine/DNA mixture was incubated on the cells for 3-4 hours and then replaced with an antibiotic-free, serum-containing growth medium. After 24 hours the cells were re-plated in selective medium containing Glutamax DMEM supplemented with 10% FBS, 0.005 mg/ml blasticidin. 0.36 mg/ml G418, 0.4 g/ml puromycin, and 0.1 mg/ml zeocin at 37.degrees C.

Resistant colonies were expanded, and identified as containing the T1R2/T1R3 sweet heterodimer by their response to various sweetener compounds including sucrose, sucralose, aspartame and acesulfame K, which was determined via automated fluorimetric imaging on the FLIPR-Tetra instrumentation (Molecular Devices) using the methods described in example 1. All potential clones were also evaluated for a functional response to sweet tastants in the presence of 10 μg/ml tetracycline (to induce overexpression of T1R2) as well as in the absence of tetracycline induction to identify any clones that are basally expressing low-level but sufficient expression of the T1R2 receptor to allow for assembly with T1R3 resulting in a functional sweet heterodimer complex (The tetracycline-regulated systems such as the T-Rex HEK-293 (Invitrogen) are known to have a low-level basal expression of transgenes due to the inherent leakiness of the system.).

As a result of said evalution, it was determined that when these cell lines, which were not treated with tetracycline, are exposed sweet tastants, many of cell clones respond to these stimuli with a significant increase in fluorescence (greater than 10-times signal in negative control cells).

Signals were lower in cells treated with tetracycline to induce overexpression of the T1R2. The lower response in the tetracycline-induced T1R2/T1R3 cells may be due to cellular toxicity arising from tetracycline-induced overexpression of the T1R2. One clonal cell line exhibiting the greatest response to sweet tastants was propagated and used for subsequent comparisons to the T1R2-TMD stable cell line.

### Example 5

Identification of Perillartine as T1R2-TMD Aqonist

Cells employed were HEK293T cells stably expressing G16gust44 and stably transfected with T1R2-TMD, which were formed as described in example 3.

Intracellular calcium responses to 50 μM perillartine were determined.

Cells were plated in black, clear-bottom plates (Costar) at a density of 8500 cells/well and maintained in selective growth media (as described in example 3) for 48 hours before determining receptor activity as described in example 1.

The cells were not induced with tetracycline since the particular clone selected already basally expressed sufficient levels of the T1R2-TMD to generate a robust increase in intracellular calcium following ligand stimulation.

The data was calculated as described in example 1 and illustrated the net increase in fluorescence over baseline following stimulation of the cells with 50 μM perillartine. The data represent the mean±Standard deviation of six replicate experiments.

Significant increases in calcium signaling were observed upon stimulation with perillartine in cells stably expressing human the T1R2-TMD but not in the negative control (host cells expressing only the G16gust44 chimeric G-protein).

| | AVERAGE | STD |
|---|---|---|
| | [RFU] | [RFU] |
| T1R2-TMD/G16gust44 | 973.06 | 69.48 |
| G16gust44 ONLY | 73.78 | 55.43 |
| (neg. control) | | |

### Example 6

Dose Response Curves of T1R2-TMD Homomer and T1R2/T1R3 Heterodimer to Perillartine

The method quantitates T1R2-TMD activity and allows, for example, to predict the potency of identified modulators including sweet tastants.

HEK293T cells that stably express G16gust44 and T1R2-TMD (formed as described in example 3) are loaded with the calcium dye Fluo-4, and their response to perillartine is measured by using fluorescent calcium signals as described in example 1. The data was calculated as described in example 1 (net increase in fluorescence over baseline following stimulation of the cells with increasing doses of perillartine, over a range of 0.1 to 200 micromolar). The data includes the mean±standard deviation (STD) of three replicate experiments.

To confirm the in vivo relevance, the dose-response curve of signals elicited by perillartine in T1R2-TMD expressing cells are compared to the signals obtained in cells that stably express the T1R2/T1R3 heterodimer. The two dose-response curves are found to match closely (see FIG. 1)

The results are shown in the table below and the dose response curves is shown in FIG. 1. The data in the table was curve-fit with the GraphPad Prism software package (GraphPad Software, Inc) using a 4-parameter logistic non-linear regression equation, shown below:

Y=Bottom+(Top−Bottom)/(1+10̂((Log EC50−X)*HillSlope)

where:
X is the logarithm of concentration. Y is the response
Y starts at Bottom and goes to Top with a sigmoid shape.

From these data the EC50 value, which represents the agonist concentration that elicits a 50% of maximum response and is indicative of receptor sensitivity (a lower EC50 value indicates greater sensitivity to an agonist), was calculated from this regression anlaysis.

The calculated EC50 value for T1R2-TMD is 6.2 micromolar and for the T1R2/T1R3 heterodimer is 3.5 micromolar.

The similar dose response and similar EC50, which is in the same sensitivity range, indicates that T1R2-TMD is a biologically relevant receptor.

| T1R2-TMD | | T1R2/T1R3 heterodimer | |
|---|---|---|---|
| AVERAGE | STD | AVERAGE | STD |
| [RFU] | [RFU] | [RFU] | [RFU] |
| 1093.24 | 64.88 | 929.14 | 68.45 |
| 1019.61 | 68.83 | 902.95 | 82.16 |
| 1010.79 | 73.23 | 874.83 | 87.04 |
| 953.94 | 63.83 | 840.69 | 87.40 |
| 768.43 | 84.59 | 730.81 | 54.59 |
| 489.68 | 45.02 | 735.04 | 100.86 |
| 399.58 | 119.53 | 385.13 | 24.38 |
| 178.86 | 68.37 | 140.21 | 38.04 |
| 61.96 | 7.16 | 45.33 | 3.76 |
| 20.53 | 4.77 | 15.15 | 7.73 |
| 33.09 | 7.03 | 28.38 | 7.75 |
| 18.18 | 2.70 | 14.90 | 2.53 |

### Example 7

Identification of Compounds that Activate T1R2-TMD but not the T1R2/T1R3 Heterodimer

Using the calcium flux assay described in example 1, a panel of 88 test agents were evaluated for T1R2-TMD receptor-dependent responses.

Test agents were tested in duplicate at a final concentration of 100 micromolar.

The signals elicited by the test agents in cells that stably express G16gust44 and T1R2-TMD containing cells (formed as described in example 3) were compared to the signals obtained in cells that stably express G16gust44 and the T1R2/T1R3 heterodimer (formed as described in example 4), and as a negative control, cells that stably express G16gust44 were used.

The data was calculated as described in example 1 (net increase in fluorescence over baseline following stimulation of the cells with test agonist) and the results of the calcium signals for the identified agents that strongly activate the T1R2-TMD but elicit little or no activation of the T1R2/T1R3 heterodimer or the negative control are shown in the table below. The data corresponds to the average of two replicates from one representative experiment, which were confirmed in subsequent tests.

For the identified compound shown in the table below, a significant increase in the calcium signal was observed upon stimulation with said compound in cells stably expressing T1R2-TMD.

Cells expressing the T1R2/T1R3 heterodimer showed no signals significantly above the negative control.

The result shows that T1R2-TMD is activated by a compound that does not activate the T1R2/T1R3 heterodimer; accordingly assays based on the T1R2-TMD homomer serve to identify modulators that cannot be identified using assays performed in presence of T1R3 and based on the T1R2/T1R3 heterodimer.

Methyl chavicol (FEMA # 2411, Estragole; p-Methoxyallylbenzene) is a known flavour described to have a sweet taste and the taste is described as follows: “sweet, herbaceous, anise-fennel odor”, “sweet, phenolic, anise, harsh, spice, green, herbal, minty” odor and a “sweet, licorice, phenolic, weedy, spice, celery-like” taste at 10 ppm.

| | T1R2 | | Negative |
|---|---|---|---|
| | TMD | T1R2/T1R3 | control |
| Methyl chavicol | [RFU] | [RFU] | [RFU] |
| 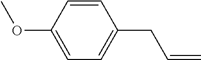 | 7372.615 | 777.765 | 732.195 |

While the sweet receptor protein, nucleic acid, methods and kit have been described above in connection with certain illustrative embodiments, it is to be understood that other similar embodiments may be used or modifications and additions may be made to the described embodiments for performing the same function. Further, all embodiments disclosed are not necessarily in the alternative, as various embodiments may be combined to provide the desired characteristics. Variations can be made by one having ordinary skill in the art without departing from the spirit and scope of the disclosure. Therefore, the method and kit should not be limited to any single embodiment, but rather construed in breadth and scope in accordance with the recitation of the attached claims.

1. A T1R2-TMD sweet receptor able to bind to and be activated by perillartine comprising one or more of
a polypeptide comprising SEQ ID NO:2,
a polypeptide sequence having at least 74% homology to SEQ. ID NO. 2 as determined by sequence identity;
a polypeptide encoded by a nucleotide sequence having at least 65% homology to SEQ ID NO:1 as determined by sequence identity,
a polypeptide encoded by a nucleotide sequence having at least 65% homology to a nucleotide sequence encoding the polypeptide set forth in SEQ ID NO:2 as determined by hybridisation,
a polypeptide encoded by a nucleotide sequence having at least 65% homology to a nucleotide sequence encoding the polypeptide set forth in SEQ ID NO:2 as determined by nucleotide sequence identity, and
wherein the substantially homologous nucleotide as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;
with the proviso that the T1R2-TMD receptor does not comprise one or more of
a polypeptide comprising SEQ ID NO:10 or SEQ ID NO:12,
a polypeptide encoded by a nucleotide sequence having at least 50% homology to a nucleotide sequence set forth in SEQ ID NO:9 or SEQ ID NO:11 as determined by sequence identity,
a polypeptide encoded by a nucleotide sequence having at least 60% homology to a nucleotide sequence encoding the polypeptide set forth in SEQ ID NO:10 or SEQ ID NO:12 as determined by hybridisation,
a polypeptide encoded by a nucleotide sequence having at least 60% homology to a nucleotide sequence encoding the polypeptide set forth in SEQ ID NO:10 or SEQ ID NO:12 as determined by nucleotide sequence identity,
wherein the nucleotide sequence having at least 50% homology to a nucleotide sequence set forth in SEQ ID NO:9 or SEQ ID NO:11 as determined by hybridisation hybridises under moderately stringent hybridization conditions at a temperature of 42° C. in a solution consisting of 50% formamide, 5×SSC, and 1% SDS, and washing at 42° C. in a solution consisting of 0.2×SSC and 0.1% SDS.

2. A nucleic acid encoding a T1R2-TMD sweet receptor that is able to bind to and be activated by perillartine comprising one or more of
a nucleic acid having at least 65% homology to a nucleotide sequence set forth in SEQ ID NO:1 as determined by sequence identity,
a nucleic acid having at least 65% homology to a nucleotide sequence set forth in SEQ ID NO:1 as determined by hybridisation,
a nucleic acid having at least 65% homology to a nucleotide sequence encoding the T1R2-TMD sweet receptor,
wherein the nucleotide having at least 65% homology to SEQ ID NO. 1 as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;
with the proviso that the nucleic acid does not comprise one or more of
a nucleic acid having at least 50% homology to a nucleotide sequence set forth in SEQ ID NO:9 or SEQ ID NO:11 as determined by sequence identity,
a nucleic acid having at least 50% homology to a nucleotide sequence set forth in SEQ ID NO:9 or SEQ ID NO:11 as determined by hybridisation,
a nucleic acid sequence encoding a polypeptide having at least 60% homology to the polypeptide of SEQ ID NO:10 or SEQ ID NO:12,
wherein the nucleotide sequence having at least 50% homology to SEQ ID NO. 9 or SEQ. ID. NO. 11 as determined by hybridisation hybridises under moderately stringent hybridization conditions at a temperature of 42° C. in a solution consisting of 50% formamide, 5×SSC, and 1% SDS, and washing at 42° C. in a solution consisting of 0.2×SSC and 0.1% SDS.

3. A T1R2-TMD sweet receptor selected from the group consisting of
a polypeptide comprising SEQ ID NO:2,
a polypeptide encoded by a nucleotide sequence having at least 65% homology to a nucleotide sequence set forth in SEQ ID NO:1 as determined by sequence identity,
a polypeptide encoded by a nucleotide sequence having at least 74% homology to a nucleotide sequence encoding the polypeptide set forth in SEQ ID NO:2 as determined by hybridisation,
a polypeptide encoded by a nucleotide sequence having at least 65% homology to a nucleotide sequence encoding the polypeptide set forth in SEQ ID NO:2 as determined by nucleotide sequence identity,
wherein the nucleotide sequence having at least 65% homology to SEQ. ID NO. 1 as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS.

4. An isolated nucleic acid selected from the group consisting of
a nucleic acid having at least 65% homology to a nucleotide sequence set forth in SEQ ID NO:1 as determined by sequence identity,
a nucleic acid having at least 65% homology to a nucleotide sequence set forth in SEQ ID NO:1 as determined by hybridisation,
a nucleic acid sequence having at least 65% homology to SEQ ID NO. 1 encoding the the polypeptide having at least 74% homology to SEQ. ID NO. 2,
wherein the nucleotide sequence having at least 65% homology to SEQ ID NO. 1 as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS.

5. An expression vector comprising the nucleic acid as defined in claim 4.

6. A host cell transfected with an expression vector as defined in claim 5, but that does not contain T1R3.

7. The host cell of claim 6 stably expressing a T1R2-TMD sweet receptor selected from the group consisting of
a polypeptide comprising SEQ ID NO:2,
a polypeptide having a homology of at least 74% to SEQ ID NO. 2 as determined by sequence identity;
a polypeptide encoded by a nucleotide sequence having at least 65% homology to a nucleotide sequence set forth in SEQ ID NO:1 as determined by sequence identity,
a polypeptide encoded by a nucleotide sequence having at least 65% homology to a nucleotide sequence encoding the polypeptide set forth in SEQ ID NO:2 as determined by hybridisation,
a polypeptide encoded by a nucleotide sequence having at least 65% homology to a nucleotide sequence encoding the polypeptide set forth in SEQ ID NO:2 as determined by nucleotide sequence identity,
wherein the nucleotide sequence having at least 65% homology to a nucleotide sequence set forth in SEQ ID NO:1 as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;
and a G-protein.

8. The host cell of claim 6 transiently expressing a T1R2-TMD sweet receptor selected from the group consisting of
a polypeptide comprising SEQ ID NO:2,
a polypeptide having a homology of at least 74% to SEQ ID NO. 2 as determined by sequence identity;
a polypeptide encoded by a nucleotide sequence having at least 65% homology to a nucleotide sequence set forth in SEQ ID NO:1 as determined by sequence identity,
a polypeptide encoded by a nucleotide sequence having at least 65% homology to a nucleotide sequence encoding the polypeptide set forth in SEQ ID NO:2 as determined by hybridisation,
a polypeptide encoded by a nucleotide sequence having at least 65% homology to a nucleotide sequence encoding the polypeptide set forth in SEQ ID NO:2 as determined by nucleotide sequence identity,
wherein the nucleotide sequence having at least 65% homology to a nucleotide sequence set forth in SEQ ID NO:1 as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS;
and a G-protein.

9. A method of producing a T1R2-TMD sweet receptor as defined in claim 1, comprising the step of culturing host cells having contained therein an expression vector encoding for the T1R2-TMD sweet receptor, under conditions sufficient for expression, thereby forming the T1R2-TMD sweet receptor and optionally recovering it from the cells.

10. A method of producing a T1R2-TMD sweet receptor as defined in claim 3, comprising the step of culturing host cells having contained therein an expression vector encoding for the T1R2-TMD sweet receptor, under conditions sufficient for expression, thereby forming the T1R2-TMD sweet receptor and optionally recovering it from the cells.

11. A method to identify an agent that modulates sweet taste signaling in taste cells comprising:
(i) providing cells that express a T1R2-TMD sweet receptor that responds to sweet taste stimuli;
(ii) contacting the cells that express a T1R2-TMD sweet receptor that responds to sweet taste stimuli with an agent, optionally in presence of another agent; and
(ii) determining whether at least one agent affects the functional activity of said T1R2-TMD sweet receptor in said cells by at least one functional response in said cells;
wherein said T1R2-TMD sweet receptor is selected from the group consisting of a polypeptide having at least 74% homology to SEQ ID NO:2, and a polypeptide encoded by a nucleotide having at least 65% homology to SEQ ID NO:1 as determined by sequence identity, and a polypeptide encoded by a nucleotide having at least 65% homology to SEQ ID NO:1 as determined by hybridisation;
wherein the nucleotide sequence having at least 65% homology to SEQ ID NO. 1 as determined by hybridisation hybridises under stringent hybridization conditions at a temperature of 42° C. in a solution consisting of 50% formamide, 5×SSC, and 1% SDS, and washing at 65° C. in a solution consisting of 0.2×SSC and 0.1% SDS; and
wherein the T1R2 sweet receptor expressing cells do not express a T1R3 receptor.

12. The method of claim 11, wherein the cells also express a G-protein.

13. The method of claim 12, wherein the G-protein is a chimeric G-protein based on Gaq-Gustducin.

14. The method of claim 12, wherein the G-protein is the chimeric G-protein G alpha 16-gustducin 44.

15. The method of claim 11, wherein (ii) is performed by measuring a change in or caused by intracellular messengers.

16. The method of claim 12, wherein the functional response is determined by measuring a change in an intracellular messenger selected from IP3 and calcium²⁺.

17. The method of claim 11, wherein said cells are selected from the group consisting of bacterial cells, eucaryotic cells, yeast cells, insect cells, mammalian cells, amphibian cells, and worm cells.

18. The method of claim 17, wherein the cells are mammalian cells.

19. The method of claim 11, wherein the cells are mammalian cells selected from the group consisting of CHO, COS, HeLa and HEK-293 cells.

20. The method of claim 11, wherein (i) further comprises contacting cells that express a T1R2-TMD sweet receptor with a test agent in presence of a sweet tastant.

21. The method of claim 20, wherein the sweet tastant is selected from the group consisting of perillartine and methyl chavicol.

22. A kit comprising:
(i) recombinant cells that express a T1R2-TMD sweet receptor, or a substantially similar homologue thereof, but that do not express a T1R3 receptor, and
(ii) an agonist of the T1R2-TMD sweet receptor,
for combined use to identify test agents as modulators of the T1R2-TMD.

23. A method of using the kit of claim 22 comprising:
(i) growing recombinant cells on a solid support;
(ii) adding test agents to a culture medium of defined plates or wells in the presence of the agonist in a suitable concentration, and
(iii) determining a change in a functional response of the cells by comparing the response in presence and absence of the test agent, and the test agent is thereby identified as a modulator of the T1R2 sweet receptor or a substantially similar homologue therof.

24. The method of claim 23, comprising adding a test agent in an amount from about 1 nM to about 100 mM to a culture medium of defined plates or wells in the presence of the agonist in a suitable concentration.

25. A method to identify an agent that modulates T1R2-TMD comprising:
(i) measuring a parameter that changes in response to a ligand binding to T1R2-TMD; and
(ii) determining a change of the parameter in response to a test agent, optionally in presence of a ligand, in comparison to a negative control and thereby identifying a modulator or ligand.

26. The method of claim 25, wherein the ligand is selected from the group consisting of perillartine and methyl chavicol.

27. The method of claim 25, wherein (i) is performed by a method selected from the group consisting of fluorescence spectroscopy, NMR spectroscopy, measuring of one or more of absorbance, refractive index, hydrodynamic methods, chromatography, measuring solubility, biochemical, wherein the methods measure the properties of the T1R2-TMD polypeptide in a suitable environment selected form the group consisting of solution, bilayer membrane, attached to a solid phase, in a lipid monolayer, bound on a membrane, and in vesicles.

28. The method of claim 26, wherein (i) is performed by a method selected from the group consisting of fluorescence spectroscopy, NMR spectroscopy, measuring of one or more of absorbance, refractive index, hydrodynamic methods, chromatography, measuring solubility, biochemical, wherein the methods measure the properties of the T1R2-TMD polypeptide in a suitable environment selected form the group consisting of solution, bilayer membrane, attached to a solid phase, in a lipid monolayer, bound on a membrane, and in vesicles.


SEQUENCE LISTING (12 sequences)

SEQ ID NO: 1 - DNA, 828 bp, Homo sapiens
  CDS (1)..(828)
gcacccaccatcgctgtggccctgctggccgccctgggcttcctcagcaccctggccatcctggtgatattctggaggcacttccagacacccatagttcgctcggctgggggccccatgtgcttcctgatgctgacactgctgctggtggcatacatggtggtcccggtgtacgtggggccgcccaaggtctccacctgcctctgccgccaggccctctttcccctctgcttcacaatttgcatctcctgtatcgccgtgcgttctttccagatcgtctgcgccttcaagatggccagccgcttcccacgcgcctacagctactgggtccgctaccaggggccctacgtctctatggcatttatcacggtactcaaaatggtcattgtggtaattggcatgctggccacgggcctcagtcccaccacccgtactgaccccgatgaccccaagatcacaattgtctcctgtaaccccaactaccgcaacagcctgctgttcaacaccagcctggacctgctgctctcagtggtgggtttcagcttcgcctacatgggcaaagagctgcccaccaactacaacgaggccaagttcatcaccctcagcatgaccttctatttcacctcatctgtctccctctgcaccttcatgtctgcctacagcggggtgctggtcaccatcgtggacctcttggtcactgtgctcaacctcctggccatcagcctgggctacttcggccccaagtgctacatgatcctcttctacccggagcgcaacacgcccgcctacttcaacagcatgatccagggctacaccatgaggagggac

SEQ ID NO: 2 - protein, 276 aa, Homo sapiens
APTIAVALLAALGFLSTLAILVIFWRHFQTPIVRSAGGPMCFLMLTLLLVAYMVVPVYVGPPKVSTCLCRQALFPLCFTICISCIAVRSFQIVCAFKMASRFPRAYSYWVRYQGPYVSMAFITVLKMVIVVIGMLATGLSPTTRTDPDDPKITIVSCNPNYRNSLLFNTSLDLLLSVVGFSFAYMGKELPTNYNEAKFITLSMTFYFTSSVSLCTFMSAYSGVLVTIVDLLVTVLNLLAISLGYFGPKCYMILFYPERNTPAYFNSMIQGYTMRRD

SEQ ID NO: 3 - DNA, 144 bp, rat
  CDS (4)..(144)
accatggccgctgttacctatccttcatccgtgcctacgaccttggaccctgggaatgcatcctcagcctggcccctggacacgtccctggggaatgcatctgctggcactagcctggcaggactggctgtcagtggcgaattc

SEQ ID NO: 4 - protein, 47 aa, rat
MAAVTYPSSVPTTLDPGNASSAWPLDTSLGNASAGTSLAGLAVSGEF

SEQ ID NO: 5 - DNA, 45 bp, herpes simplex
  CSD (1)..(45)
  CDS (1)..(45)
tgcggccgccagcctgaactcgctcctgaagacccggaagattaa

SEQ ID NO: 6 - protein, 14 aa, herpes simplex
CGRQPELAPEDPED

SEQ ID NO: 7 - DNA, 31 bp, Artificial
  synthesized primer sequence
tatagaattcgcacccaccatcgctgtggcc

SEQ ID NO: 8 - DNA, 28 bp, artificial
  synthesized primer sequence
atatgcggccgcagtccctcctcatggt

SEQ ID NO: 9 - DNA, 2520 bp, Homo sapiens
  CDS (1)..(2520)
atggggcccagggcaaagaccatctcctccctgttcttcctcctatgggtcctggctgagccggctgagaactcggacttctacctgcctggggattacctcctgggtggcctcttctccctccatgccaacatgaagggcattgttcaccttaacttcctgcaggtgcccatgtgcaaggagtatgaagtgaaggtgataggctacaacctcatgcaggccatgcgctttgcggtggaggagatcaacaatgacagcagcctgctgcctggtgtgctgctgggctatgagatcgtggatgtgtgctacatctccaacaatgtccagccggtgctctacttcctggcacacgaggacaacctccttcccatccaagaggactacagtaactacatttcccgtgtggtggctgtcattggccctgacaactccgagtctgtcatgactgtggccaacttcctctccctatttctccttccacagatcacctacagcgccatcagcgatgagctgcgagacaaggtgcgcttcccggctttgctgcgtaccacacccagcgccgaccaccacatcgaggccatggtgcagctgatgctgcacttccgctggaactggatcattgtgctggtgagcagcgacacctatggccgcgacaatggccagctgcttggcgagcgcgtggcccggcgcgacatctgcatcgccttccaggagacgctgcccacactgcagcccaaccagaacatgacgtcagaggagcgccagcgcctggtgaccattgtggacaagctgcagcagagcacagcgcgcgtcgtggtcgtgttctcgcccgacctgaccctgtaccacttcttcaatgaggtgctgcgccagaacttcactggcgccgtgtggatcgcctccgagtcctgggccatcgacccggtcctgcacaacctcacggagctgcgccacttgggcaccttcctgggcatcaccatccagagcgtgcccatcccgggcttcagtgagttccgcgagtggggcccacaggctgggccgccacccctcagcaggaccagccagagctatacctgcaaccaggagtgcgacaactgcctgaacgccaccttgtccttcaacaccattctcaggctctctggggagcgtgtcgtctacagcgtgtactctgcggtctatgctgtggcccatgccctgcacagcctcctcggctgtgacaaaagcacctgcaccaagagggtggtctacccctggcagctgcttgaggagatctggaaggtcaacttcactctcctggaccaccaaatcttcttcgacccgcaaggggacgtggctctgcacttggagattgtccagtggcaatgggaccggagccagaatcccttccagagcgtcgcctcctactaccccctgcagcgacagctgaagaacatccaagacatctcctggcacaccatcaacaacacgatccctatgtccatgtgttccaagaggtgccagtcagggcaaaagaagaagcctgtgggcatccacgtctgctgcttcgagtgcatcgactgccttcccggcaccttcctcaaccacactgaagatgaatatgaatgccaggcctgcccgaataacgagtggtcctaccagagtgagacctcctgcttcaagcggcagctggtcttcctggaatggcatgaggcacccaccatcgctgtggccctgctggccgccctgggcttcctcagcaccctggccatcctggtgatattctggaggcacttccagacacccatagttcgctcggctgggggccccatgtgcttcctgatgctgacactgctgctggtggcatacatggtggtcccggtgtacgtggggccgcccaaggtctccacctgcctctgccgccaggccctctttcccctctgcttcacaatctgcatctcctgtatcgccgtgcgttctttccagatcgtctgcgccttcaagatggccagccgcttcccacgcgcctacagctactgggtccgctaccaggggccctacgtctctatggcatttatcacggtactcaaaatggtcattgtggtaattggcatgctggccacgggcctcagtcccaccacccgtactgaccccgatgaccccaagatcacaattgtctcctgtaaccccaactaccgcaacagcctgctgttcaacaccagcctggacctgctgctctcagtggtgggtttcagcttcgcctacatgggcaaagagctgcccaccaactacaacgaggccaagttcatcaccctcagcatgaccttctatttcacctcatctgtctccctctgcaccttcatgtctgcctacagcggggtgctggtcaccatcgtggacctcttggtcactgtgctcaacctcctggccatcagcctgggctacttcggccccaagtgctacatgatcctcttctacccggagcgcaacacgcccgcctacttcaacagcatgatccagggctacaccatgaggagggactag

SEQ ID NO: 10 - protein, 839 aa, Homo sapiens
MGPRAKTISSLFFLLWVLAEPAENSDFYLPGDYLLGGLFSLHANMKGIVHLNFLQVPMCKEYEVKVIGYNLMQAMRFAVEEINNDSSLLPGVLLGYEIVDVCYISNNVQPVLYFLAHEDNLLPIQEDYSNYISRVVAVIGPDNSESVMTVANFLSLFLLPQITYSAISDELRDKVRFPALLRTTPSADHHIEAMVQLMLHFRWNWIIVLVSSDTYGRDNGQLLGERVARRDICIAFQETLPTLQPNQNMTSEERQRLVTIVDKLQQSTARVVVVFSPDLTLYHFFNEVLRQNFTGAVWIASESWAIDPVLHNLTELRHLGTFLGITIQSVPIPGFSEFREWGPQAGPPPLSRTSQSYTCNQECDNCLNATLSFNTILRLSGERVVYSVYSAVYAVAHALHSLLGCDKSTCTKRVVYPWQLLEEIWKVNFTLLDHQIFFDPQGDVALHLEIVQWQWDRSQNPFQSVASYYPLQRQLKNIQDISWHTINNTIPMSMCSKRCQSGQKKKPVGIHVCCFECIDCLPGTFLNHTEDEYECQACPNNEWSYQSETSCFKRQLVFLEWHEAPTIAVALLAALGFLSTLAILVIFWRHFQTPIVRSAGGPMCFLMLTLLLVAYMVVPVYVGPPKVSTCLCRQALFPLCFTICISCIAVRSFQIVCAFKMASRFPRAYSYWVRYQGPYVSMAFITVLKMVIVVIGMLATGLSPTTRTDPDDPKITIVSCNPNYRNSLLFNTSLDLLLSVVGFSFAYMGKELPTNYNEAKFITLSMTFYFTSSVSLCTFMSAYSGVLVTIVDLLVTVLNLLAISLGYFGPKCYMILFYPERNTPAYFNSMIQGYTMRRD

SEQ ID NO: 11 - DNA, 2559 bp, Homo sapiens
  CDS (1)..(2559)
atgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccggtgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 12 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE